(19) United States
(12) Patent Application Publication
(10) Pub. No.: US 2021/0347810 A1
(43) Pub. Date: Nov. 11, 2021

(54) STEROL PURIFICATION

(71) Applicant: ModernaTX, Inc., Cambridge, MA (US)

(72) Inventors: Gabor BUTORA, Cambridge, MA (US); Jin LIM, Cambridge, MA (US); Gary SHEN, Cambridge, MA (US); Alison VOLKERT, Cambridge, MA (US)

(21) Appl. No.: 17/278,095

(22) PCT Filed: Sep. 19, 2019
(86) PCT No.: PCT/US2019/051940

§ 371 Date: Mar. 19, 2021

Related U.S. Application Data
(60) Provisional application No. 62/733,522, filed on Sep. 19, 2018.

(51) Int. Cl. C07J 9/00; A61K 9/51; C07J 17/00; C07J 31/00; C07J 33/00; C07J 41/00; C07J 43/00; A61K 31/575; A61K 45/06

(52) U.S. Cl. CPC C07J 9/00; A61K 9/5123; C07J 17/00; C07J 31/006; C07J 33/002; C07J 41/0088; B82Y 5/00; C07J 43/003; A61K 9/5192; A61K 31/575; A61K 9/5146; A61K 45/06; C07J 41/0055

(57) ABSTRACT

The invention relates to sterol esters and methods for producing purified sterols that can be utilized in methods for producing a lipid nanoparticle.

### BACKGROUND OF THE INVENTION

In recent years, nucleic acids have increasingly been looked to as possible therapeutic agents. Therapeutic uses of messenger ribonucleic acid (mRNA) are particularly sought as an mRNA could be designed to encode a wide variety of polypeptides for many applications. For example, many diseases, disorders, and conditions, including cystic fibrosis, are characterized by aberrant protein activity and/or protein deficiency. It is theorized that the introduction of an appropriate mRNA could be translated within a cell to generate a polypeptide to replace, subvert, or otherwise combat an aberrant species. mRNA delivery systems could also be used to regulate important polypeptides such as vascular endothelial growth factor (VEGF), the transient and targeted expression of which is posited to combat stenosis in renovascular structures. Disruption of translational machineries by the introduction of non-translatable mRNA may also be feasible. However, the delivery of therapeutic RNAs to cells is made difficult by the relative instability and low cell permeability of RNAs.

Accordingly, there exists a need to develop methods and lipid-containing compositions to facilitate the delivery of RNAs such as mRNA to cells, especially with regards to improvements in safety, efficacy, and specificity.

### SUMMARY OF THE INVENTION

The invention features sterol esters and synthetic methods for producing purified β-sitosterol, campesterol, and sitostanol that may be utilized in methods for producing lipid nanoparticles (e.g., for delivery of RNA).

In an aspect, the invention features a compound having the structure of Formula I:

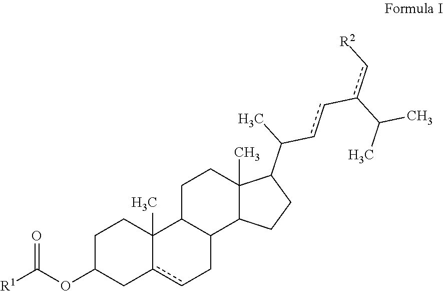

wherein the dotted lines represent optional double bonds;

R¹ is trifluoromethyl, trichloromethyl, iso-propyl, tert-butyl, 4-methyl-phenyl, 4-carboxylic acid-phenyl, 3-carboxylic acid-propyl, optionally substituted C₂-C₉ heteroaryl, optionally substituted C₁-C₆ alkyl C₂-C₉ heteroaryl, optionally substituted C₁-C₆ alkyl C₂-C₉ heterocyclyl, optionally substituted C₁-C₆ heteroalkyl, optionally substituted C₃-C₈ cycloalkyl, optionally substituted C₆-C₂₁ alkyl, or optionally substituted C₁-C₂₁ alkenyl; and

R² is hydrogen or methyl;

or a pharmaceutically acceptable salt thereof.

In some embodiments, the compound has the structure:

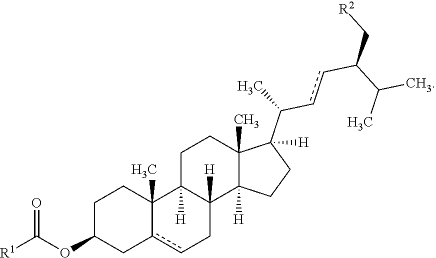

In some embodiments, the compound has the structure:

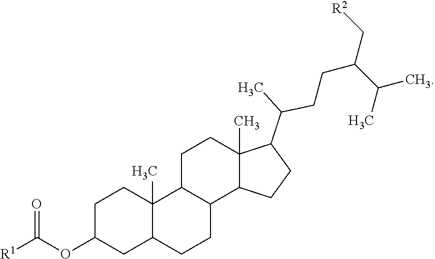

In some embodiments, the compound has the structure:

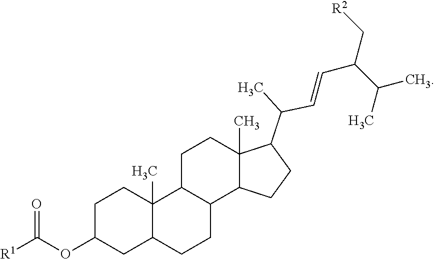

In some embodiments, the compound has the structure:

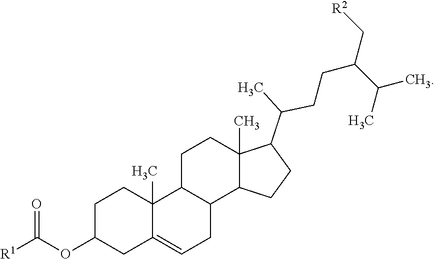

In some embodiments, the compound has the structure:

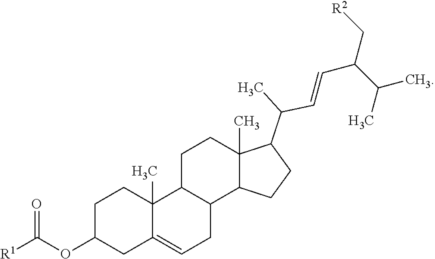

In some embodiments, the compound has the structure:

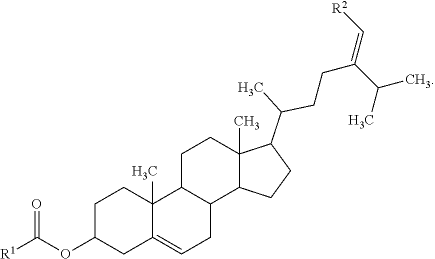

In some embodiments, the compound has the structure:

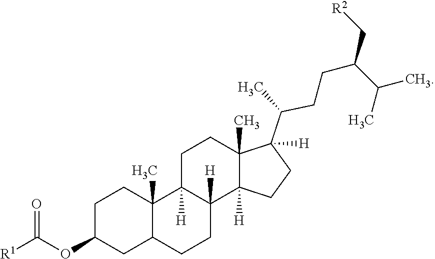

In some embodiments, the compound has the structure:

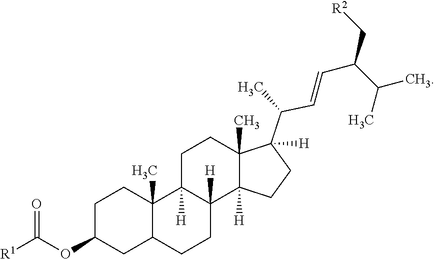

In some embodiments, the compound has the structure:

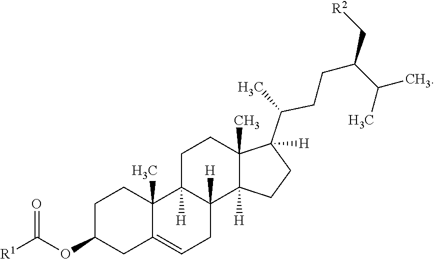

In some embodiments, the compound has the structure:

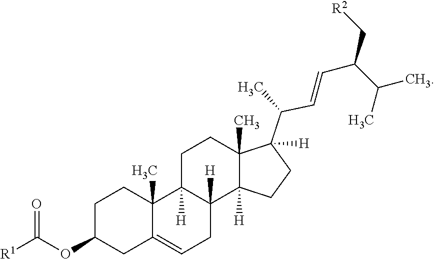

In some embodiments, R² is hydrogen. In some embodiments, R² is methyl.

In some embodiments, R¹ is trifluoromethyl. In some embodiments, R¹ is trichloromethyl. In some embodiments, R¹ is iso-propyl. In some embodiments, R¹ is tert-butyl. In some embodiments, R¹ is 4-methyl-phenyl. In some embodiments, R¹ is 4-carboxylic acid-phenyl. In some embodiments, R¹ is 3-carboxylic acid-propyl. In some embodiments, R¹ is optionally substituted C₂-C₉ heteroaryl. In some embodiments, R¹ is optionally substituted C₁-C₆ alkyl C₂-C₉ heteroaryl. In some embodiments, R¹ is optionally substituted C₁-C₆ alkyl C₂-C₉ heterocyclyl. In some embodiments, R¹ is optionally substituted C₁-C₆ heteroalkyl. In some embodiments, R¹ is optionally substituted C₃-C₈ cycloalkyl. In some embodiments, R¹ is optionally substituted C₆-C₂₁ alkyl. In some embodiments, R¹ is optionally substituted C₁-C₂₁ alkenyl. In some embodiments, R¹ is —(CH₂)₁₆CH₃, —CF₃, —CCl₃, cyclopropyl, tert-butyl, iso-propyl, 4-methyl-phenyl,

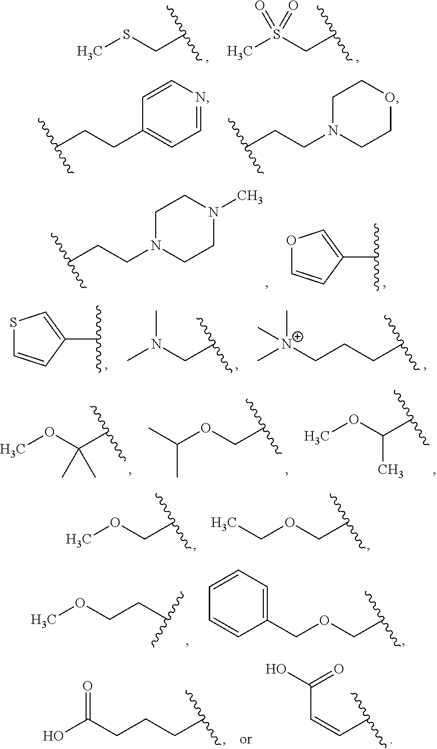

In some embodiments, the compound is any one of compounds 1 to 33 in Table 1.

TABLE 1
Compounds of the Invention

| Com- | |
|---|---|
| pound | |
| No. | Structure |
| 1 | 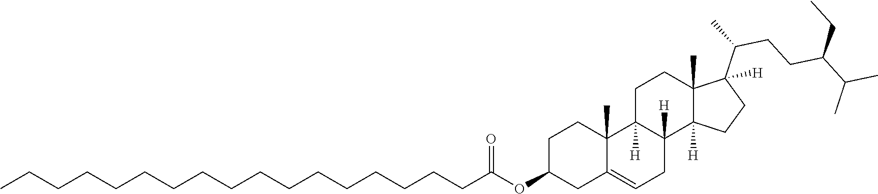 |
| 2 | 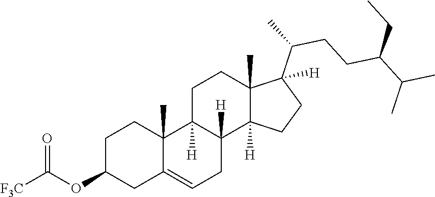 |
| 3 | 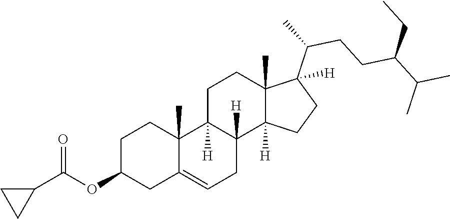 |
| 4 | 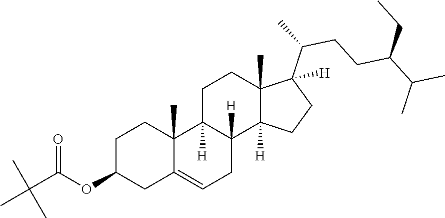 |
| 5 | 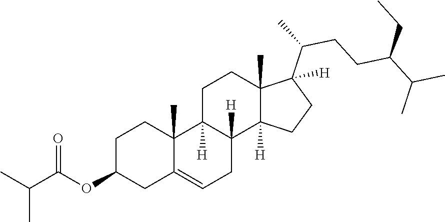 |
| 6 | 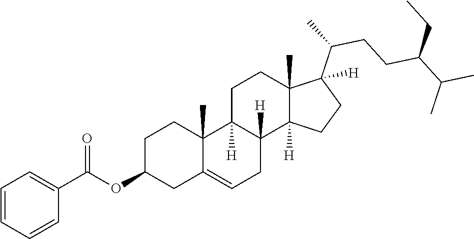 |
| 7 | 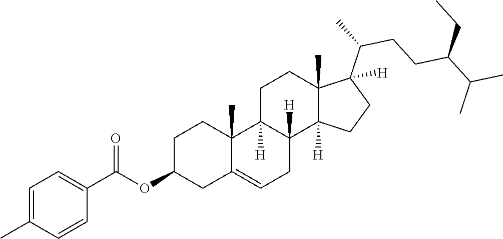 |
| 8 | 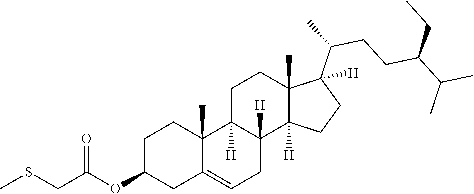 |
| 9 | 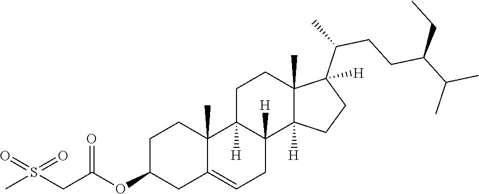 |
| 10 | 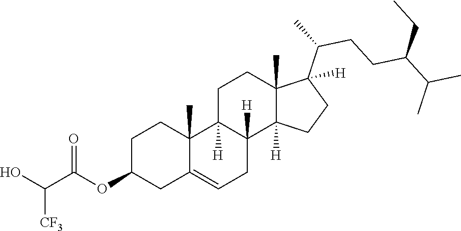 |
| 11 | 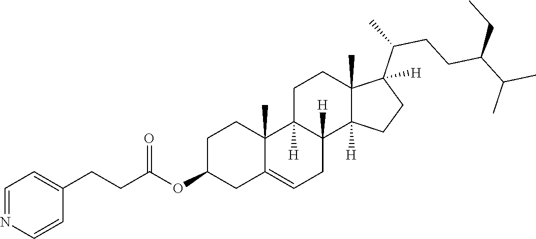 |
| 12 | 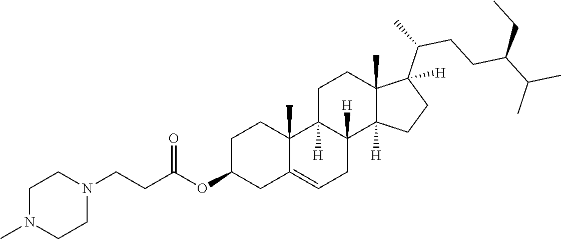 |
| 13 | 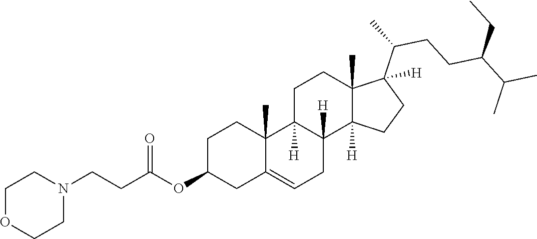 |
| 14 | 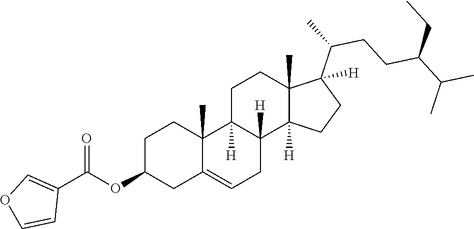 |
| 15 | 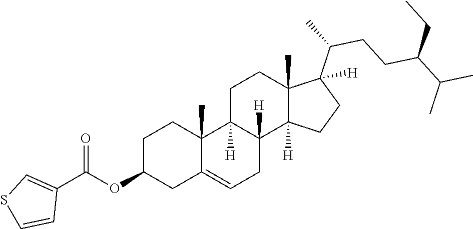 |
| 16 | 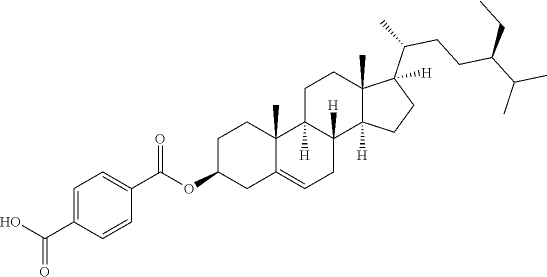 |
| 17 | 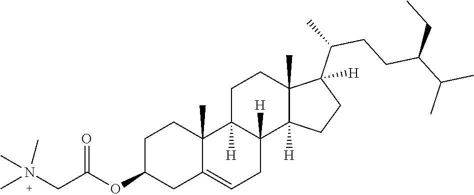 |
| 18 | 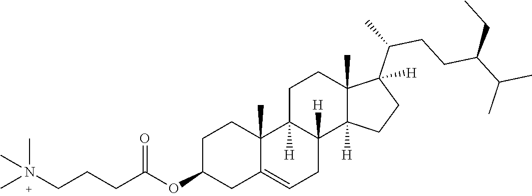 |
| 19 | 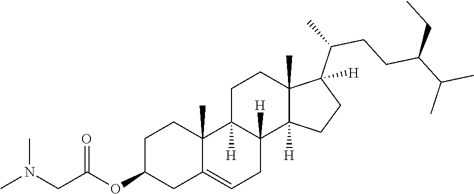 |
| 20 | 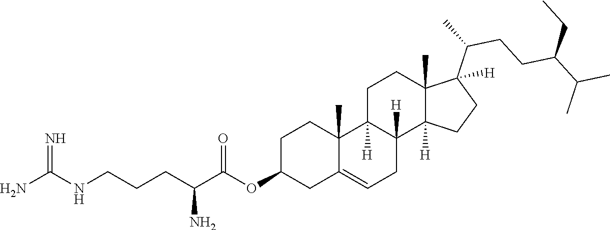 |
| 21 | 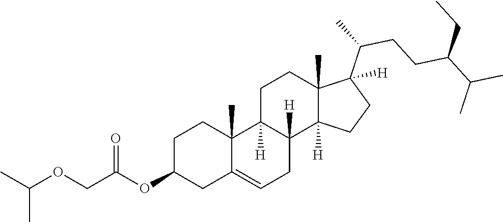 |
| 22 | 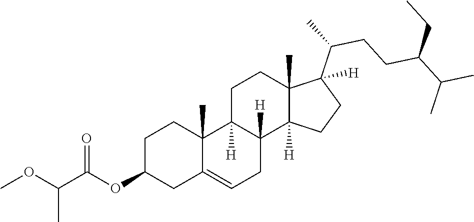 |
| 23 | 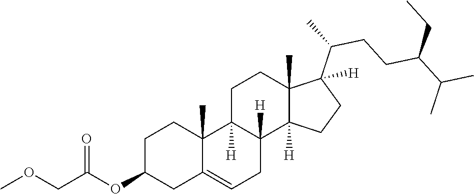 |
| 24 | 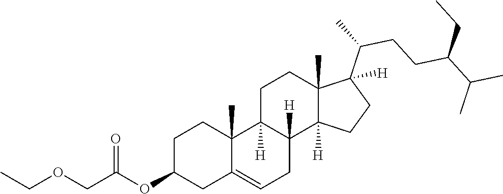 |
| 25 | 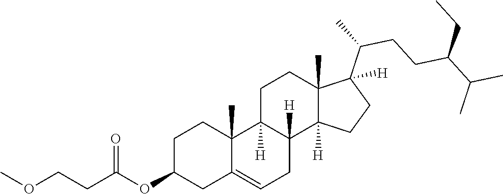 |
| 26 | 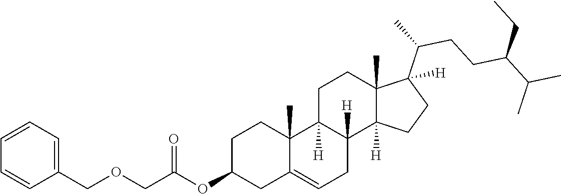 |
| 27 | 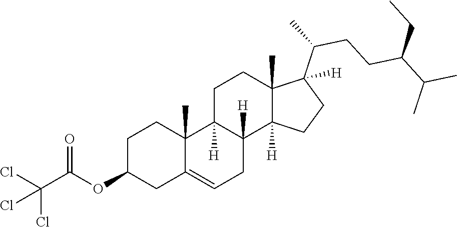 |
| 28 | 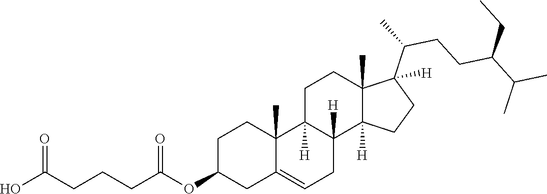 |
| 29 | 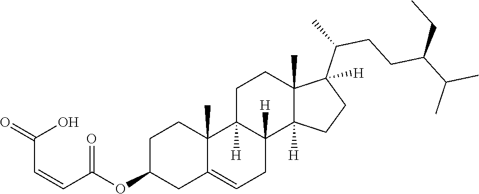 |
| 30 | 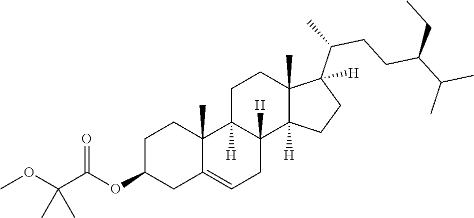 |
| 31 | 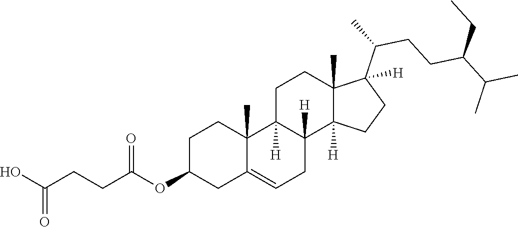 |
| 32 | 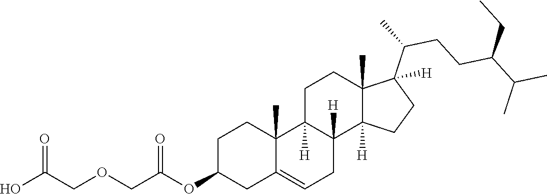 |
| 33 | 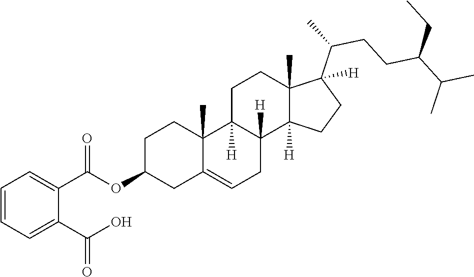 |

In another aspect, the invention features a composition including a compound of any of the foregoing compounds and an excipient.

In another aspect, the invention features a method of producing purified β-sitosterol, the method including:

(a) obtaining a sample including β-sitosterol and one or more other sterols;

(b) reacting the sample under conditions sufficient to produce a β-sitosterol ester;

(c) separating the β-sitosterol ester from the one or more other sterols in the sample to produce a sample of β-sitosterol ester; and

(d) reacting the sample of β-sitosterol ester under conditions sufficient to hydrolyze the β-sitosterol ester, thereby producing purified β-sitosterol.

In some embodiments, the method further includes: (e) recrystallizing the product produced in step (d).

In some embodiments, the β-sitosterol ester has the structure of Formula II:

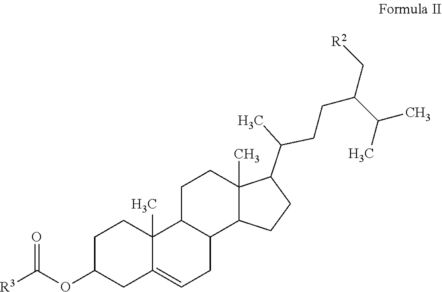

wherein R³ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,

or a pharmaceutically acceptable salt thereof.

In some embodiments, R³ is C₂-C₉ heteroaryl. In some embodiments, R³ is optionally substituted C₁-C₆ alkyl C₂-C₉ heteroaryl. In some embodiments, R³ is optionally substituted C₆-C₁₀ aryl. In some embodiments, R³ is optionally substituted C₁-C₆ alkyl C₆-C₁₀ aryl. In some embodiments, R³ is optionally substituted C₁-C₆ alkyl C₂-C₉ heterocyclyl. In some embodiments, R³ is optionally substituted C₁-C₆ heteroalkyl. In some embodiments, R³ is optionally substituted C₃-C₈ cycloalkyl. In some embodiments, R³ is optionally substituted C₁-C₂₁ alkyl. In some embodiments, R³ is optionally substituted C₁-C₂₁ alkenyl. In some embodiments, R³ is —(CH₂)₁₆CH₃, —CF₃, —CCl₃, cyclopropyl, tert-butyl, iso-propyl, phenyl, 4-methyl-phenyl,

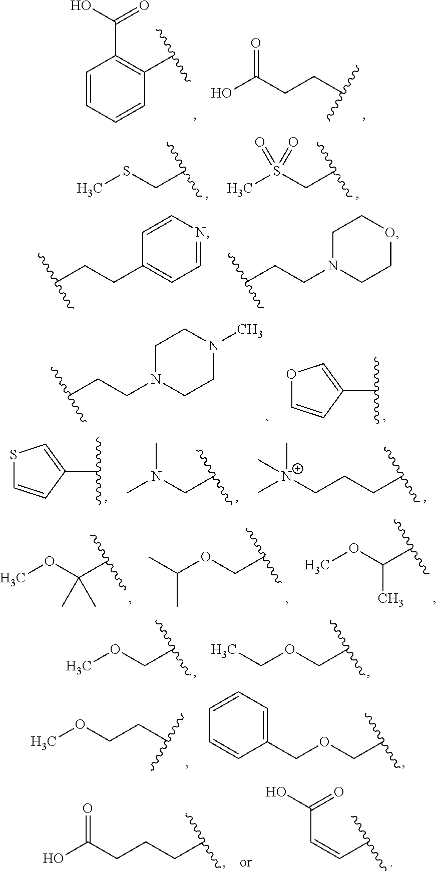

In another aspect, the invention features a method of producing purified campesterol, the method including:

(a) obtaining a sample including campesterol and one or more other sterols;

(b) reacting the sample under conditions sufficient to produce a campesterol ester;

(c) substantially separating the campesterol ester from the one or more other sterols in the sample to produce a sample of campesterol ester; and

(d) reacting the sample of campesterol ester under conditions sufficient to hydrolyze the campesterol ester,

thereby producing purified campesterol.

In some embodiments, the method further includes: (e) recrystallizing the product produced in step (d).

In some embodiments, the campesterol ester has the structure of Formula III:

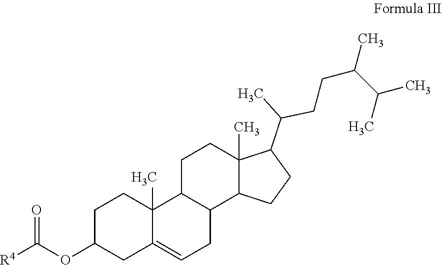

wherein R⁴ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,

or a pharmaceutically acceptable salt thereof.

In some embodiments, R⁴ is C₂-C₉ heteroaryl. In some embodiments, R⁴ is optionally substituted C₁-C₆ alkyl C₂-C₉ heteroaryl. In some embodiments, R⁴ is optionally substituted C₆-C₁₀ aryl. In some embodiments, R⁴ is optionally substituted C₁-C₆ alkyl C₆-C₁₀ aryl. In some embodiments, R⁴ is optionally substituted C₁-C₆ alkyl C₂-C₉ heterocyclyl. In some embodiments, R⁴ is optionally substituted C₁-C₆ heteroalkyl. In some embodiments, R⁴ is optionally substituted C₃-C₈ cycloalkyl. In some embodiments, R⁴ is optionally substituted C₁-C₂₁ alkyl. In some embodiments, R⁴ is optionally substituted C₁-C₂₁ alkenyl. In some embodiments, R⁴ is —(CH₂)₁₆CH₃, —CF₃, —CCl₃, cyclopropyl, tert-butyl, iso-propyl, phenyl, 4-methyl-phenyl,

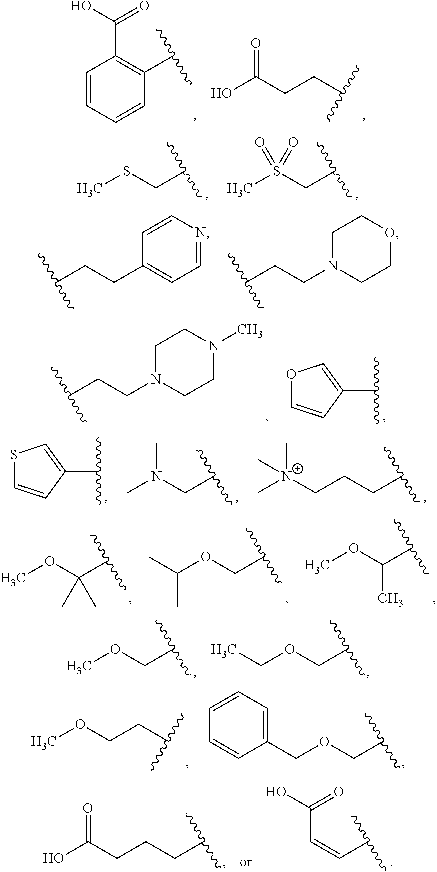

In another aspect, the invention features a method of producing purified sitostanol, the method including:

(a) obtaining a sample including sitostanol and one or more other sterols;

(b) reacting the sample under conditions sufficient to produce a sitostanol ester;

(c) separating the sitostanol ester from the one or more other sterols in the sample to produce a sample of sitostanol ester; and

(d) reacting the sample of sitostanol ester under conditions sufficient to hydrolyze the sitostanol ester, thereby producing purified sitostanol.

In some embodiments, the method further includes: (e) recrystallizing the product produced in step (d).

In some embodiments, the sitostanol ester has the structure of Formula IV:

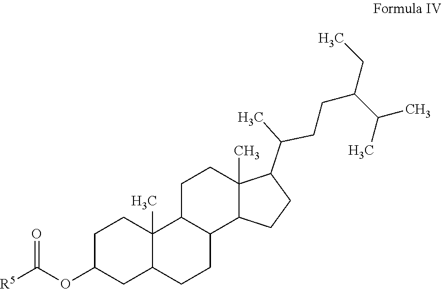

wherein R⁵ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl, or a pharmaceutically acceptable salt thereof.

In some embodiments, R⁵ is C₂-C₉ heteroaryl. In some embodiments, R⁵ is optionally substituted C₁-C₆ alkyl C₂-C₉ heteroaryl. In some embodiments, R⁵ is optionally substituted C₆-C₁₀ aryl. In some embodiments, R⁵ is optionally substituted C₁-C₆ alkyl C₆-C₁₀ aryl. In some embodiments, R⁵ is optionally substituted C₁-C₆ alkyl C₂-C₉ heterocyclyl. In some embodiments, R⁵ is optionally substituted C₁-C₆ heteroalkyl. In some embodiments, R⁵ is optionally substituted C₃-C₈ cycloalkyl. In some embodiments, R⁵ is optionally substituted C₁-C₂₁ alkyl. In some embodiments, R⁵ is optionally substituted C₁-C₂₁ alkenyl. In some embodiments, R⁵ is —(CH₂)₁₆CH₃, —CF₃, —CCl₃, cyclopropyl, tert-butyl, iso-propyl, phenyl, 4-methyl-phenyl,

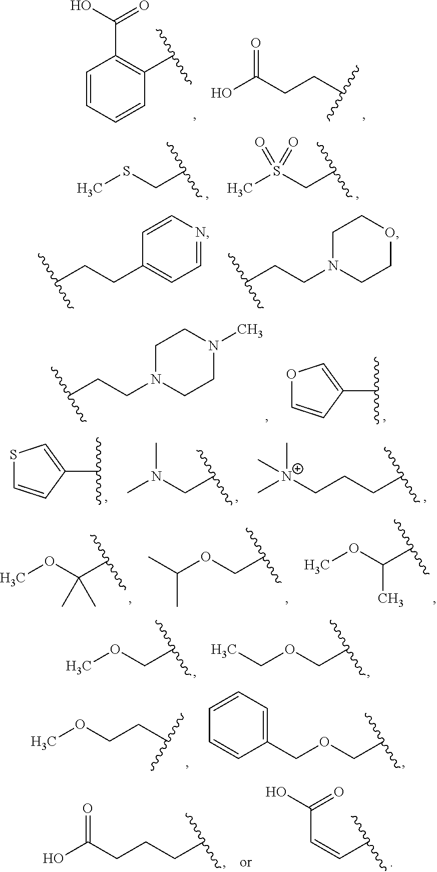

In another aspect, the invention features a method of producing purified stigmasterol, the method including:

(a) obtaining a sample including stigmasterol and one or more other sterols;

(b) reacting the sample under conditions sufficient to produce a stigmasterol ester;

(c) separating the stigmasterol ester from the one or more other sterols in the sample to produce a sample of stigmasterol ester; and

(d) reacting the sample of stigmasterol ester under conditions sufficient to hydrolyze the stigmasterol ester, thereby producing purified stigmasterol.

In some embodiments, the method further includes: (e) recrystallizing the product produced in step (d).

In some embodiments, the stigmasterol ester has the structure of Formula V:

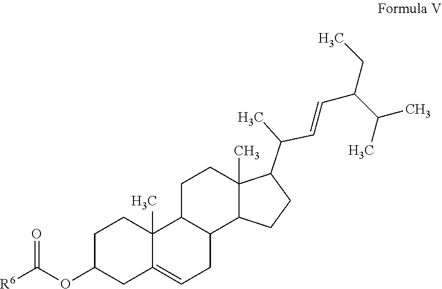

wherein R⁶ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl, or a pharmaceutically acceptable salt thereof.

In some embodiments, R⁶ is C₂-C₉ heteroaryl. In some embodiments, R⁶ is optionally substituted C₁-C₆ alkyl C₂-C₉ heteroaryl. In some embodiments, R⁶ is optionally substituted C₆-C₁₀ aryl. In some embodiments, R⁶ is optionally substituted C₁-C₆ alkyl C₆-C₁₀ aryl. In some embodiments, R⁶ is optionally substituted C₁-C₆ alkyl C₂-C₉ heterocyclyl. In some embodiments, R⁶ is optionally substituted C₁-C₆ heteroalkyl. In some embodiments, R⁶ is optionally substituted C₃-C₈ cycloalkyl. In some embodiments, R⁶ is optionally substituted C₁-C₂₁ alkyl. In some embodiments, R⁶ is optionally substituted C₁-C₂₁ alkenyl. In some embodiments, R⁶ is —(CH₂)₁₆CH₃, —CF₃, —CCl₃, cyclopropyl, tert-butyl, iso-propyl, phenyl, 4-methyl-phenyl,

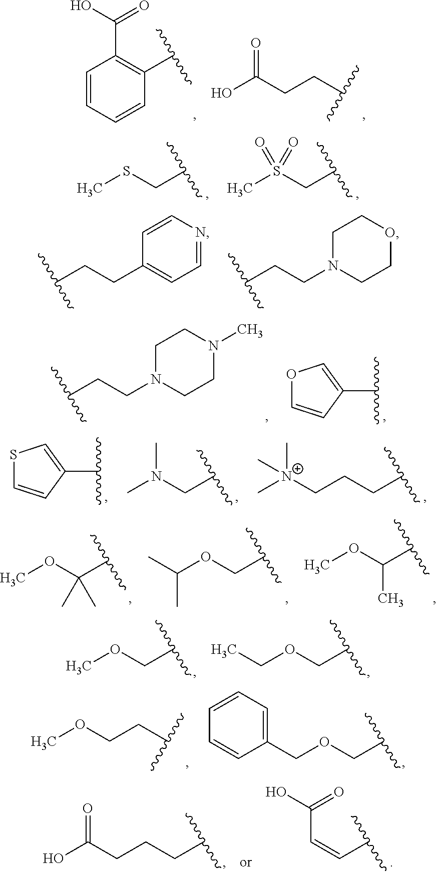

In another aspect, the invention features a method of producing purified campestanol, the method including:

(a) obtaining a sample including campestanol and one or more other sterols;

(b) reacting the sample under conditions sufficient to produce a campestanol ester;

(c) separating the campestanol ester from the one or more other sterols in the sample to produce a sample of campestanol ester; and

(d) reacting the sample of campestanol ester under conditions sufficient to hydrolyze the campestanol ester, thereby producing purified campestanol.

In some embodiments, the method further includes: (e) recrystallizing the product produced in step (d).

In some embodiments, the campestanol ester has the structure of Formula VI:

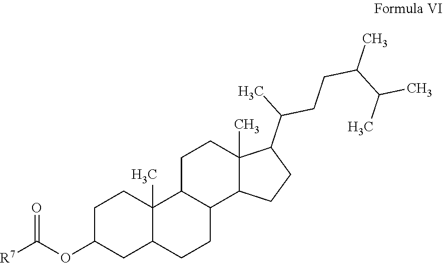

wherein R⁷ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl, or a pharmaceutically acceptable salt thereof.

In some embodiments, R⁷ is C₂-C₉ heteroaryl. In some embodiments, R⁷ is optionally substituted C₁-C₆ alkyl C₂-C₉ heteroaryl. In some embodiments, R⁷ is optionally substituted C₆-C₁₀ aryl. In some embodiments, R⁷ is optionally substituted C₁-C₆ alkyl C₆-C₁₀ aryl. In some embodiments, R⁷ is optionally substituted C₁-C₆ alkyl C₂-C₉ heterocyclyl. In some embodiments, R⁷ is optionally substituted C₁-C₆ heteroalkyl. In some embodiments, R⁷ is optionally substituted C₃-C₈ cycloalkyl. In some embodiments, R⁷ is optionally substituted C₁-C₂₁ alkyl. In some embodiments, R⁷ is optionally substituted C₁-C₂₁ alkenyl. In some embodiments, R⁷ is —(CH₂)₁₆CH₃, —CF₃, —CCl₃, cyclopropyl, tert-butyl, iso-propyl, phenyl, 4-methyl-phenyl,

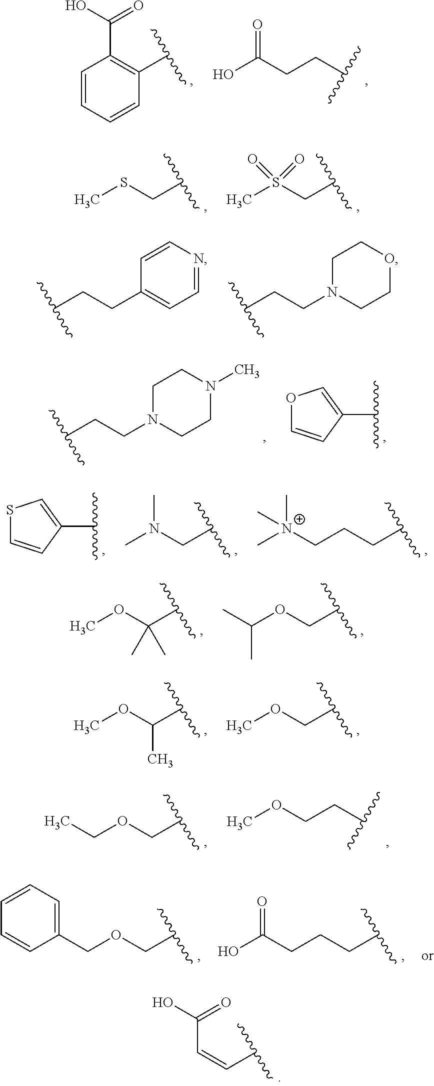

In another aspect, the invention features a method of producing purified brassicasterol, the method including:

(a) obtaining a sample including brassicasterol and one or more other sterols;

(b) reacting the sample under conditions sufficient to produce a brassicasterol ester;

(c) separating the brassicasterol ester from the one or more other sterols in the sample to produce a sample of brassicasterol ester; and

(d) reacting the sample of brassicasterol ester under conditions sufficient to hydrolyze the brassicasterol ester,

thereby producing purified brassicasterol.

In some embodiments, the method further includes: (e) recrystallizing the product produced in step (d).

In some embodiments, the brassicasterol ester has the structure of Formula VII:

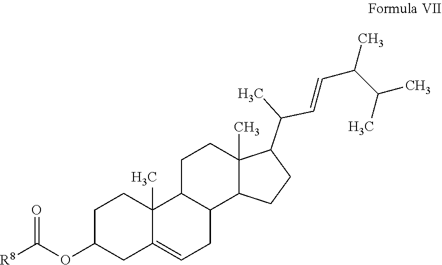

wherein R⁸ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,

or a pharmaceutically acceptable salt thereof.

In some embodiments, R⁸ is C₂-C₉ heteroaryl. In some embodiments, R⁸ is optionally substituted C₁-C₆ alkyl C₂-C₉ heteroaryl. In some embodiments, R⁸ is optionally substituted C₆-C₁₀ aryl. In some embodiments, R⁸ is optionally substituted C₁-C₆ alkyl C₆-C₁₀ aryl. In some embodiments, R⁸ is optionally substituted C₁-C₆ alkyl C₂-C₉ heterocyclyl. In some embodiments, R⁸ is optionally substituted C₁-C₆ heteroalkyl. In some embodiments, R⁸ is optionally substituted C₃-C₈ cycloalkyl. In some embodiments, R⁸ is optionally substituted C₁-C₂₁ alkyl. In some embodiments, R⁸ is optionally substituted C₁-C₂₁ alkenyl. In some embodiments, R⁸ is —(CH₂)₁₆CH₃, —CF₃, —CCl₃, cyclopropyl, tert-butyl, iso-propyl, phenyl, 4-methyl-phenyl,

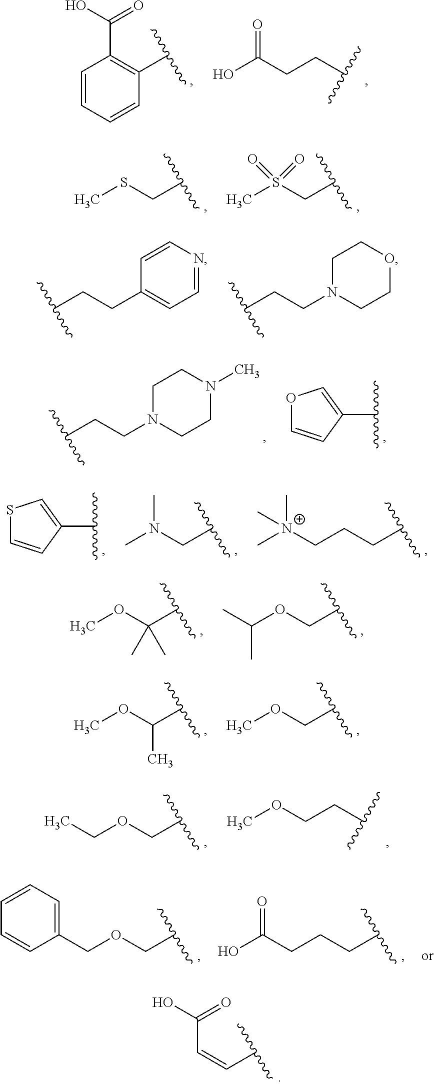

In another aspect, the invention features a method of producing purified fucosterol, the method including:

(a) obtaining a sample including fucosterol and one or more other sterols;

(b) reacting the sample under conditions sufficient to produce a fucosterol ester;

(c) separating the fucosterol ester from the one or more other sterols in the sample to produce a sample of fucosterol ester; and

(d) reacting the sample of fucosterol ester under conditions sufficient to hydrolyze the fucosterol ester, thereby producing purified fucosterol.

In some embodiments, the method further includes: (e) recrystallizing the product produced in step (d).

In some embodiments, the fucosterol ester has the structure of Formula VIII:

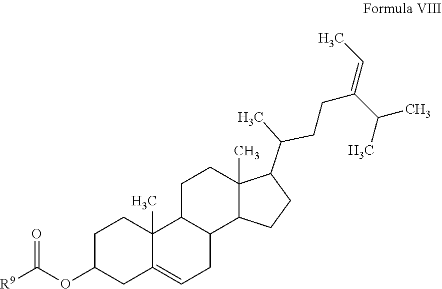

wherein R⁹ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl, or a pharmaceutically acceptable salt thereof.

In some embodiments, R⁹ is C₂-C₉ heteroaryl. In some embodiments, R⁹ is optionally substituted C₁-C₆ alkyl C₂-C₉ heteroaryl. In some embodiments, R⁹ is optionally substituted C₆-C₁₀ aryl. In some embodiments, R⁹ is optionally substituted C₁-C₆ alkyl C₆-C₁₀ aryl. In some embodiments, R⁹ is optionally substituted C₁-C₆ alkyl C₂-C₉ heterocyclyl. In some embodiments, R⁹ is optionally substituted C₁-C₆ heteroalkyl. In some embodiments, R⁹ is optionally substituted C₃-C₈ cycloalkyl. In some embodiments, R⁹ is optionally substituted C₁-C₂₁ alkyl. In some embodiments, R⁹ is optionally substituted C₁-C₂₁ alkenyl. In some embodiments, R⁹ is —(CH₂)₁₆CH₃, —CF₃, —CCl₃, cyclopropyl, tert-butyl, iso-propyl, phenyl, 4-methyl-phenyl,

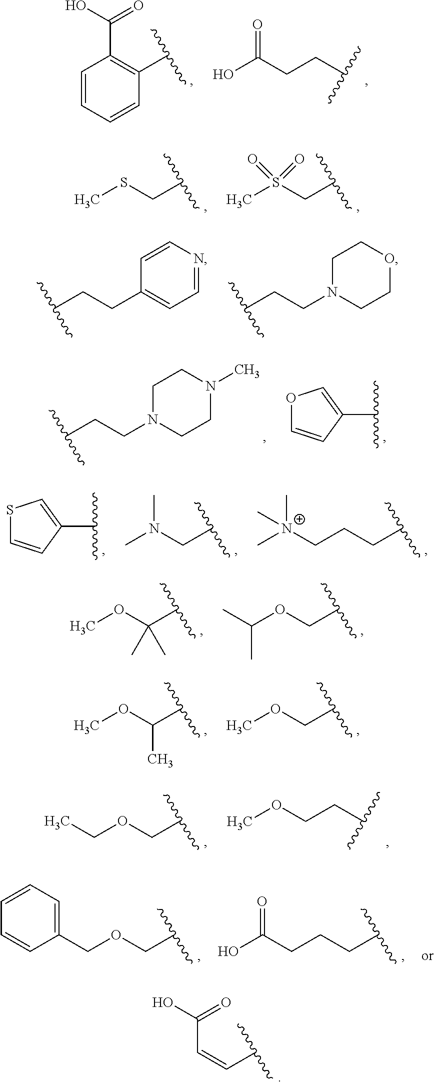

In some embodiments of any of the foregoing methods, the conditions sufficient to produce a β-sitosterol ester, campesterol ester, sitostanol ester, stigmasterol ester, campestanol ester, fucosterol ester, or brassicasterol ester include (i) an acid anhydride and a base; (ii) a carboxylic acid and a carboxyl activating agent; or (iii) an acyl chloride.

In some embodiments of any of the foregoing methods, the conditions sufficient to produce a β-sitosterol ester, campesterol ester, sitostanol ester, stigmasterol ester, campestanol ester, fucosterol ester, or brassicasterol ester include an acid anhydride and a base (e.g., an organic base such as triethylamine).

In some embodiments of any of the foregoing methods, the conditions sufficient to produce a β-sitosterol ester, campesterol ester, sitostanol ester, stigmasterol ester, campestanol ester, fucosterol ester, or brassicasterol ester include a carboxylic acid and a carboxyl activating agent (e.g., -Ethyl-3-(3-dimethylaminopropyl)carbodiimide).

In some embodiments of any of the foregoing methods, the conditions sufficient to produce a β-sitosterol ester, campesterol ester, sitostanol ester, stigmasterol ester, campestanol ester, fucosterol ester, or brassicasterol ester include an acyl chloride.

In some embodiments of any of the foregoing methods, the conditions sufficient to produce a β-sitosterol ester, campesterol ester, sitostanol ester, stigmasterol ester, campestanol ester, fucosterol ester, or brassicasterol ester further include 4-(dimethylamino)pyridine.

In some embodiments of any of the foregoing methods, the separating includes normal phase purification. In some embodiments of any of the foregoing methods, the separating includes reverse phase purification.

In some embodiments of any of the foregoing methods, the conditions sufficient to hydrolyze the β-sitosterol ester, campesterol ester, sitostanol ester, stigmasterol ester, campestanol ester, fucosterol ester, or brassicasterol ester include water, an alcohol (e.g., 2-propanol) and a base (e.g., an inorganic base such as sodium hydroxide or potassium hydroxide).

In some embodiments of any of the foregoing methods, the purified sample of β-sitosterol ester, campesterol ester, sitostanol ester, stigmasterol ester, campestanol ester, fucosterol ester, or brassicasterol ester has a purity of at least 90% (e.g., at least 95%, at least 99%, at least 99.5%).

In another aspect, the invention features a method of producing a lipid nanoparticle, the method including

(i) preparing purified β-sitosterol, campesterol, sitostanol, stigmasterol, campestanol, fucosterol, and/or brassicasterol by the method of any one of claims 9 to 34; and

(ii) contacting the purified β-sitosterol, campesterol, sitostanol, stigmasterol, campestanol, fucosterol, and/or brassicasterol and an ionizable lipid under conditions sufficient to form a lipid nanoparticle,

thereby producing a lipid nanoparticle.

In some embodiments, the method further includes contacting the β-sitosterol, campesterol, sitostanol, stigmasterol, campestanol, fucosterol, and/or brassicasterol and the ionizable lipid with a non-ionizable helper lipid and/or a PEG-lipid.

In some embodiments, the method further includes contacting the lipid nanoparticle with an mRNA encoding a polypeptide under conditions sufficient for the lipid nanoparticle to encapsulate the mRNA.

In another aspect, the invention features a composition including two or more sterols, wherein the two or more sterols include β-sitosterol and campesterol, wherein β-sitosterol includes 95-99.9% of the sterols in the composition and campesterol includes 0.1-5% of the sterols in the composition.

In some embodiments, the composition further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including two or more sterols, wherein the two or more sterols include β-sitosterol and sitostanol, wherein β-sitosterol includes 95-99.9% of the sterols in the composition and sitostanol includes 0.1-5% of the sterols in the composition.

In some embodiments, the composition further includes campesterol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and campesterol and β-sitosterol includes 95-99.9% of the sterols in the composition and campesterol includes 0.1-5% of the sterols in the composition.

In some embodiments, the two or more sterols further includes sitostanol. In some embodiments, β-sitosterol includes 95-99.9%, campesterol includes 0.05-4.95%, and sitostanol includes 0.05-4.95% of the sterols in the composition.

In another aspect, the invention features a composition including a plurality of lipid nanoparticles, wherein the plurality of lipid nanoparticles include an ionizable lipid and two or more sterols, wherein the two or more sterols include β-sitosterol, and sitostanol and β-sitosterol includes 95-99.9% of the sterols in the composition and sitostanol includes 0.1-5% of the sterols in the composition.

In some embodiments, the two or more sterols further includes campesterol. In some embodiments, β-sitosterol includes 95-99.9%, campesterol includes 0.05-4.95%, and sitostanol includes 0.05-4.95% of the sterols in the composition.

In some embodiments, the plurality of lipid nanoparticles further include a non-ionizable helper lipid and/or a PEG-lipid.

In an aspect, the invention features a lipid nanoparticle including:

(i) an ionizable lipid; and

(ii) a structural component,

wherein the structural component includes a compound having the structure of any of the foregoing compounds.

In some embodiments, the lipid nanoparticle further includes a nucleic acid molecule.

In an aspect, the invention features a lipid nanoparticle including:

(i) an ionizable lipid;

(ii) a structural component;

(iii) optionally, a non-cationic helper lipid;

(iv) optionally, a PEG-lipid; and

(v) a nucleic acid molecule,

wherein the structural component includes a compound having the structure of any of the foregoing compounds and optionally a structural lipid.

In some embodiments, the lipid nanoparticle includes the compound of any of the foregoing compounds in an amount that enhances delivery of the nucleic acid molecule to a cell relative to a lipid nanoparticle lacking said compound.

In some embodiments, the structural component further includes one or more structural lipids or salts thereof.

In some embodiments, the one or more structural lipids is a sterol.

In some embodiments, the one or more structural lipids is a phytosterol. In some embodiments, the phytosterol is β-sitosterol, campesterol, sitostanol, stigmasterol, campestanol, fucosterol, or brassicasterol or any combination thereof. In some embodiments, the phytosterol is β-sitosterol.

In some embodiments, the one or more structural lipids is a zoosterol. In some embodiments, the zoosterol is cholesterol.

In some embodiments, the mol % of the one or more structural lipids is between about 1% and 50% of the mol % of the compound having the structure of any of the foregoing compounds present in the lipid nanoparticle.

In some embodiments, the mol % of the one or more structural lipids is between about 10% and 40% of the mol % of the compound having the structure of any of the foregoing compounds present in the lipid nanoparticle.

In some embodiments, the mol % of the one or more structural lipids is between about 20% and 30% of the mol % of the compound having the structure of any of the foregoing compounds present in the lipid nanoparticle.

In some embodiments, the mol % of the one or more structural lipids is about 30% of the mol % of the compound having the structure of any of the foregoing compounds present in the lipid nanoparticle.

In some embodiments, the lipid nanoparticle includes one or more non-cationic helper lipids.

In some embodiments, the one or more non-cationic helper lipids is a phospholipid, fatty acid, or any combination thereof.

In some embodiments, the phospholipid is a phospholipid that includes a phosphocholine moiety, a phosphoethanolamine moiety, or a phosphor-1-glycerol moiety.

In some embodiments, the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, or 1,2-didocosahexaenoyl-sn-glycero-3-phosphocholine.

In some embodiments, the phospholipid is DSPC.

In some embodiments, the phospholipid is 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, or 1,2-dioleoyl-sn-glycero-3-phospho-rac-(1-glycerol) sodium salt (DOPG).

In some embodiments, the phospholipid is sphingomyelin.

In some embodiments, the fatty acid is a long-chain fatty acid. In some embodiments, the fatty acid is a very long-chain fatty acid. In some embodiments, the fatty acid is a medium-chain fatty acid.

In some embodiments, the fatty acid is palmitic acid, stearic acid, palmitoleic acid, or oleic acid.

In some embodiments, the fatty acid is oleic acid. In some embodiments, the fatty acid is stearic acid.

In some embodiments, the lipid nanoparticle includes one or more PEG-lipids.

In some embodiments, the one or more PEG-lipids is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or mixtures thereof.

In some embodiments, the one or more PEG-lipids is PEG-c-DOMG, PEG-DMG, PEG-DLPE, PEG-DMPE, PEG-DPPC, or PEG-DSPE lipid.

In some embodiments, the one or more PEG-lipids is PEG-DMG.

In some embodiments, the lipid nanoparticle includes about 30 mol % to about 60 mol % ionizable lipid or ionizable lipids, about 0 mol % to about 30 mol % to about 60 mol % one or more ionizable lipids, about 0 mol % to about 30 mol % one or more non-cationic helper lipids, about 18.5 mol % to about 48.5 mol % structural component, and about 0 mol % to about 10 mol % one or more PEG-lipids.

In some embodiments, the lipid nanoparticle includes about 35 mol % to about 55 mol % one or more ionizable lipids, about 5 mol % to about 25 mol % one or more non-cationic helper lipids, about 30 mol % to about 40 mol % structural component, and about 0 mol % to about 10 mol % one or more PEG-lipids.

In some embodiments, the lipid nanoparticle includes about 50 mol % one or more ionizable lipids, about 10 mol % one or more non-cationic helper lipids, about 38.5 mol % structural component, and about 1.5 mol % one or more PEG-lipids.

In some embodiments, the nucleic acid molecule is RNA or DNA.

In some embodiments, the nucleic acid is DNA.

In some embodiments, the nucleic acid molecule is ssDNA. In some embodiments, the nucleic acid is DNA including CRISPR.

In some embodiments, the nucleic acid is RNA.

In some embodiments, the nucleic acid molecule is a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

In some embodiments, the nucleic acid molecule is an mRNA.

In some embodiments, the mRNA is a modified mRNA including one or more modified nucleobases.

In some embodiments, the mRNA includes one or more of a stem loop, a chain terminating nucleoside, a polyA sequence, a polyadenylation signal, and a 5′ cap structure.

In some embodiments, the structural component includes a compound of Formula I. In some embodiments, the structural component includes a compound of Formula II. In some embodiments, the structural component includes a compound of Formula Ill. In some embodiments, the structural component includes a compound of Formula IV. In some embodiments, the structural component includes a compound of Formula V. In some embodiments, the structural component includes a compound of Formula VI. In some embodiments, the structural component includes a compound of Formula VII. In some embodiments, the structural component includes a compound of Formula VIII.

In some embodiments, the structural component includes a compound having the structure of any one of compounds 1-33 in Table 1.

In some embodiments, the lipid nanoparticle further includes an additional compound having the structure of any one of the foregoing compounds.

### Definitions

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a composition, “about” may mean+7-10% of the recited value. For instance, a composition including a lipid component having about 40% of a given compound may include 30-50% of the compound.

As used herein, the term “chromatography” is meant a method for separating a mixture (e.g., a mixture of compounds). The method includes a “mobile phase” and a “stationary phase.” The mixture (e.g., the mixture of compounds) is dissolved in the mobile phase. The mobile phase carries the mixture through a stationary phase. When the mobile phase is a liquid, the chromatography method is referred to as “liquid chromatography” or “LC.” In some embodiments, the liquid chromatography utilizes high pressure and is referred to as “high-performance liquid chromatography” or “HPLC.”

As used herein, the term “compound” is meant to include all geometric isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

As used herein, the term “contacting” means establishing a physical connection between two or more entities. For example, contacting a mammalian cell with a composition means that the mammalian cell and a nanoparticle are made to share a physical connection. Methods of contacting cells with external entities both in vivo and ex vivo are well known in the biological arts. For example, contacting a composition and a mammalian cell disposed within a mammal may be performed by varied routes of administration (e.g., intravenous, intramuscular, intradermal, and subcutaneous) and may involve varied amounts of compositions. Moreover, more than one mammalian cell may be contacted by a composition.

As used herein, the term “delivering” means providing an entity to a destination. For example, delivering an mRNA to a subject may involve administering a composition including the mRNA to the subject (e.g., by an intravenous, intramuscular, intradermal, or subcutaneous route). Administration of a composition to a mammal or mammalian cell may involve contacting one or more cells with the composition.

As used herein, “encapsulation” may refer to complete, substantial, or partial enclosure, confinement, surrounding, or encasement.

As used herein, an “mRNA” refers to a messenger ribonucleic acid that may be naturally or non-naturally occurring. For example, an mRNA may include modified and/or nonnaturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide of interest. Translation of an mRNA, for example, in vivo translation of an mRNA inside a mammalian cell, may produce a polypeptide of interest.

As used herein, “non-cationic helper lipid” refers to a lipid including at least one fatty acid chain including at least 8 carbon atoms (e.g., 8, 9, 10, 11, 12, 13, 14, 15, 16, 17, 18, 19, or 20 carbon atoms) and at least one polar head group moiety. In some embodiments the non-cationic helper lipid is a phospholipid or a phospholipid substitute. In some embodiments, the non-cationic helper lipid is a DSPC analog, a DSPC substitute, oleic acid, or an oleic acid analog.

As used herein, a “PEG lipid” or “PEGylated lipid” refers to a lipid comprising a polyethylene glycol component.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable excipient” refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch, glycolate, sorbitol, starch (corn), stearic acid, sucrose, talc, titanium dioxide, vitamin A, vitamin E (alpha-tocopherol), vitamin C, xylitol, and other species disclosed herein.

As used herein, “pharmaceutically acceptable salts” refers to derivatives of the disclosed compounds wherein the parent compound is altered by converting an existing acid or base moiety to its salt form (e.g., by reacting the free base group with a suitable organic acid). Compositions of the invention may also include pharmaceutically acceptable salts of one or more compounds. Pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 30 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the term “polypeptide” or “polypeptide of interest” refers to a polymer of amino acid residues typically joined by peptide bonds that can be produced naturally (e.g., isolated or purified) or synthetically.

As used herein, “size” or “mean size” in the context of compositions refers to the mean diameter of a composition.

As used herein, “sterol” refers to the subgroup of steroids also known as steroid alcohols, including a salt or ester thereof. Sterols are usually divided into two classes: 1) plant sterol (e.g., phytosterol); and 2) animal sterol (e.g., zoosterol). Zoosterols include, but are not limited to, cholesterol.

As used herein, “stanol” refers to the class of saturated sterols having no double bonds in the sterol ring structure.

The term “therapeutic agent” refers to any agent that, when administered to a subject, has a therapeutic, diagnostic, and/or prophylactic effect and/or elicits a desired biological and/or pharmacological effect.

As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the “zeta potential” is the electrokinetic potential of a lipid e.g., in a particle composition.

### Chemical Terms

It is to be understood that the terminology employed herein is for the purpose of describing particular embodiments and is not intended to be limiting.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “aryl,” as used herein, refers to an aromatic mono- or polycarbocyclic radical of 6 to 12 carbon atoms having at least one aromatic ring. Aryl groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁₋₆ alkyl C₆₋₁₀ aryl, C₁₋₁₀ alkyl C₆₋₁₀ aryl, or C₁₋₂₀ alkyl C₆₋₁₀ aryl), such as, benzyl and phenethyl. In some embodiments, the akyl and the aryl each can be further substituted with 1, 2, 3, or 4 substituent groups as defined herein for the respective groups.

The terms “carbocyclyl,” as used herein, refer to a non-aromatic C₃-C₂₀ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, monovalent mono- or polycarbocyclic radical of three to twenty, preferably three to ten or three to six carbon atoms. This term is further exemplified by radicals such as cyclopropyl, cyclobutyl, cyclopentyl, cyclohexyl, cycloheptyl, and adamantyl.

The term “cycloalkenyl,” as used herein, refers to an unsaturated, non-aromatic, monovalent mono- or polycarbocyclic radical of three to twenty, preferably, three to ten or three to six carbon atoms. This term is further exemplified by radicals such as cyclobutenyl, cyclopentenyl, cyclohexenyl, cycloheptenyl, and norbornyl.

The term “polycycloalkyl” mean a structure consisting of two or more cycloalkyl moieties that have two or more atoms in common. If the cycloalkyl moieties have exactly two atoms in common they are said to be “fused.” If the cycloalkyl moieties have more than two atoms in common they are said to be “bridged.”

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Heteroalkyl groups include, but are not excluded to, “alkoxy” which, as used herein, refers alkyl-O— (e.g., methoxy and ethoxy). A heteroalkylene is a divalent heteroalkyl group.

The term “heterocyclyl,” as used herein, denotes a mono- or polycyclic radical having 3 to 12 atoms having at least one ring containing one, two, three, or four ring heteroatoms selected from N, O or S, wherein no ring is aromatic. Heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁₋₆ alkyl C₂₋₉ heterocyclyl, C₁₋₁₀ alkyl C₂₋₉ heterocyclyl, or C₁₋₂₀ alkyl C₂₋₉ heterocyclyl). In some embodiments, the akyl and the heterocyclyl each can be further substituted with 1, 2, 3, or 4 substituent groups as defined herein for the respective groups.

The term “hydroxyl,” as used herein, represents an —OH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### DETAILED DESCRIPTION OF THE INVENTION

Provided herein are sterol esters and synthetic methods for producing purified β-sitosterol, campesterol, and sitostanol that may be utilized in methods for producing a lipid nanoparticle.

### Sterol Esters

Compounds of the invention include compounds having a structure according to Formula I:

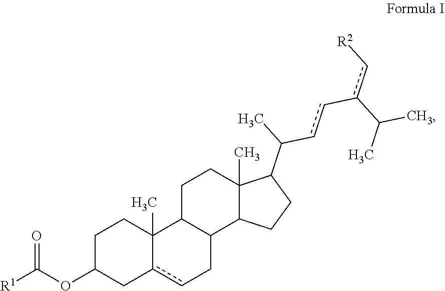

or a pharmaceutically acceptable salt thereof. Exemplary compounds of Formula I are compounds 1-33 in Table 1.

Purified β-Sitosterol, Campesterol, and Sitostanol

A method for preparing purified β-sitosterol includes obtaining a sample including β-sitosterol and one or more other sterols, producing a β-sitosterol ester, substantially separating β-sitosterol ester from the one or more other sterols in the sample to produce a sample of β-sitosterol ester, and reacting the β-sitosterol ester under conditions sufficient to hydrolyze the β-sitosterol ester to produce purified β-sitosterol.

The β-sitosterol ester can have the structure of Formula II:

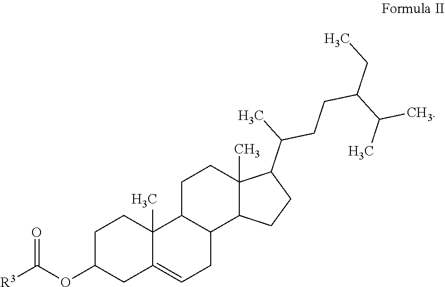

A method for preparing purified campesterol includes obtaining a sample including campesterol and one or more other sterols, producing a campesterol ester, substantially separating campesterol ester from the one or more other sterols in the sample to produce a sample of campesterol ester, and reacting the campesterol ester under conditions sufficient to hydrolyze the campesterol ester to produce purified campesterol.

The campesterol ester can have the structure of Formula III:

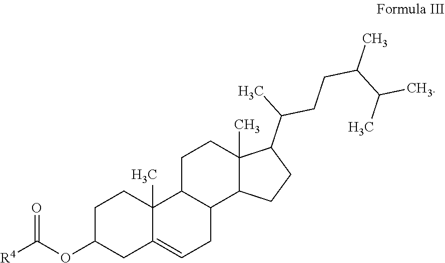

A method for preparing purified sitostanol includes obtaining a sample including sitostanol and one or more other sterols, producing a sitostanol ester, substantially separating sitostanol ester from the one or more other sterols in the sample to produce a sample of sitostanol ester, and reacting the sitostanol ester under conditions sufficient to hydrolyze the sitostanol ester to produce purified sitostanol.

The sitostanol ester can have the structure of Formula IV:

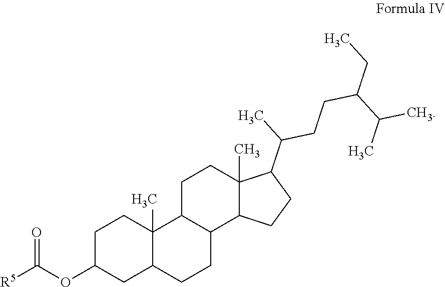

A method for preparing purified stigmasterol includes obtaining a sample including stigmasterol and one or more other sterols, producing a stigmasterol ester, substantially separating stigmasterol ester from the one or more other sterols in the sample to produce a sample of stigmasterol ester, and reacting the stigmasterol ester under conditions sufficient to hydrolyze the stigmasterol ester to produce purified stigmasterol.

The stigmasterol ester can have the structure of Formula V:

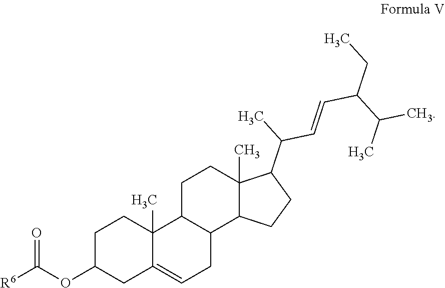

A method for preparing purified campestanol includes obtaining a sample including campestanol and one or more other sterols, producing a campestanol ester, substantially separating campestanol ester from the one or more other sterols in the sample to produce a sample of campestanol ester, and reacting the campestanol ester under conditions sufficient to hydrolyze the campestanol ester to produce purified campestanol.

The campestanol ester can have the structure of Formula VI:

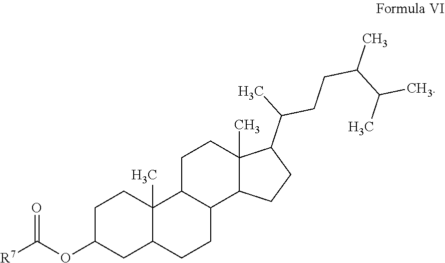

A method for preparing purified brassicasterol includes obtaining a sample including brassicasterol and one or more other sterols, producing a brassicasterol ester, substantially separating brassicasterol ester from the one or more other sterols in the sample to produce a sample of brassicasterol ester, and reacting the brassicasterol ester under conditions sufficient to hydrolyze the brassicasterol ester to produce purified brassicasterol.

The brassicasterol ester can have the structure of Formula VII:

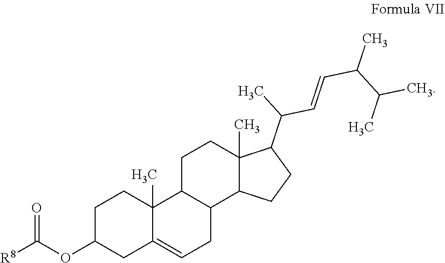

A method for preparing purified fucosterol includes obtaining a sample including fucosterol and one or more other sterols, producing a fucosterol ester, substantially separating fucosterol ester from the one or more other sterols in the sample to produce a sample of fucosterol ester, and reacting the fucosterol ester under conditions sufficient to hydrolyze the fucosterol ester to produce purified fucosterol.

The fucosterol ester can have the structure of Formula VII:

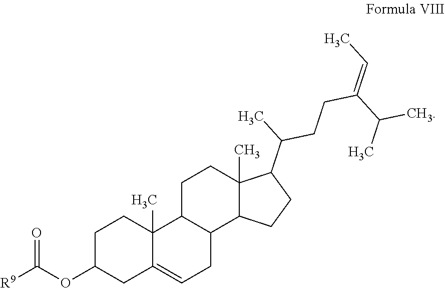

In some embodiments, substantially separating a compound (e.g., a β-sitosterol ester, a campesterol ester, or a sitostanol ester) from a mixture including said compound and one or more compounds (e.g., one or more sterols) includes separating each compound from the mixture by utilizing chromatography.

In some embodiments, the chromatography is liquid chromatography.

In some embodiments, the liquid chromatography is reverse phase. In some embodiments, the liquid chromatography is normal phase.

In some embodiments, the mobile phase includes one or more organic solvents.

In some embodiments, the mobile phase includes water.

In some embodiments, the mobile phase includes a constant ratio of one or more solvents. For example, the mobile phase can include one solvent (e.g., dichloromethane, hexanes, or methanol). The mobile phase can include a 1:1, 1:2, 1:3, 1:4, 2:3, or 1:5 ratio of two solvents (e.g., water and acetonitrile; hexanes and ethyl acetate; dichloromethane and diethyl ether; or water and methanol). In yet another example, the mobile phase can include three solvents (e.g., water, acetonitrile, and methanol; or hexanes, dichloromethane, and ethyl acetate) in a 1:1:1, 1:1:2, 1:1:3, or 1:1:4 ratio.

In some embodiments, the mobile phase includes a gradient of two or more solvents. For example, the mobile phase can include two solvents (e.g., water and acetonitrile; hexanes and ethyl acetate; dichloromethane and diethyl ether; or water and methanol) where the ratio gradually changes from 0:1 to 1:9 to 1:4 to 3:7 to 2:3 to 1:1 to 3:2 to 7:3 to 4:1 to 9:1 to 1:0 as the chromatography method advances.

In some embodiments, the one or more solvents includes one or more additives (e.g., trifluoroacetic acid, acetic acid, or triethylamine).

In some embodiments, the liquid chromatography is high pressure liquid chromatography.

In some embodiments, substantially separating a compound (e.g., a β-sitosterol ester, a campesterol ester, or a sitostanol ester) from a mixture including said compound and one or more compounds (e.g., one or more sterols) includes separating each compound from the mixture by utilizing crystallization.

### Lipid Nanoparticles

This invention features sterol esters which, in one aspect, may be utilized in lipid-containing compositions and/or in a method of synthesizing lipid-containing compositions for delivering mRNA into cells. Lipid-containing compositions have proven effective as transport vehicles into cells and/or intracellular compartments for a variety of RNAs. These compositions generally include one or more “cationic” and/or ionizable lipids, structural lipids (e.g., sterols or sterol analogs), and lipids containing polyethylene glycol (PEG-lipids). Cationic and/or ionizable lipids include, for example, amine-containing lipids that can be readily protonated.

### Ionizable Lipids

The lipid nanoparticles of the invention include one or more ionizable lipids. For example, lipid nanoparticles include an ionizable lipid. The ionizable lipids described herein may be advantageously used in lipid nanoparticles of the invention for the delivery of nucleic acid molecules to a cell (e.g., mammalian cell).

Ionizable lipids include, but are not limited to, 3-(didodecylamino)-N1,N1,4-tridodecyl-1-piperazineethanamine (KL10), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA),1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]pro pan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]prop an-1-amine (Octyl-CLinDMA (2S)). In addition to these, an ionizable lipid may also be a lipid including a cyclic amine.

Ionizable lipids include, but are not limited to, the ionizable lipids disclosed in International Publication No. WO 2015/199952, WO 2017/075531, and/or WO 2017/049245.

Ionizable lipids can have a positive or partial positive charge at physiological pH. Such ionizable lipids can be referred to as cationic and/or ionizable lipids. Ionizable lipids can be zwitterionic.

In some embodiments, ionizable lipids have the following structure:

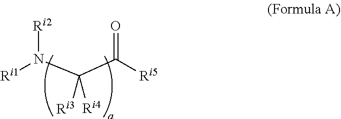

in which Rⁱ¹ is H or optionally substituted C₃-C₁₀ alkyl; each of Rⁱ² and Rⁱ⁵ is, independently, optionally substituted C₃-C₅₀ alkyl, optionally substituted C₃-C₅₀ heteroalkyl, or optionally substituted C₃-C₅₀ alkenyl; each of Rⁱ³ and Rⁱ⁴ is, independently, H or C₃-C₁₀ alkyl; and a is an integer between 5-20, or salts thereof. Examples of ionizable lipids having a structure according to Formula A include:

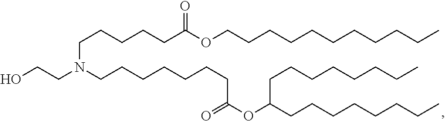 [SMILES: CCCCCCCCCCCOC(=O)CCCCCN(CCO)CCCCCCCC(=O)OC(CCCCCCCC)CCCCCCCC]

or a salt thereof.

In addition to the ionizable lipids disclosed herein, the lipid nanoparticles disclosed herein include β-sitosterol, campesterol, and/or sitostanol purified by any of the foregoing methods described in this invention. The lipid nanoparticles disclosed herein can optionally include a non-cationic helper lipid, a PEG-lipid, a structural lipid, and/or a nucleic acid molecule, or any combination thereof.

Structural Lipids

The lipid nanoparticles of the invention can include one or more structural lipids. For example, lipid nanoparticles can include a structural lipid or one or more structural lipids (e.g., two or more structural lipids, three or more structural lipids, four or more structural lipids, or five or more structural lipids). The structural lipids described herein may be advantageously used in lipid nanoparticles of the invention for the delivery of nucleic acid molecules to a cell (e.g., mammalian cell).

As used herein, “structural lipid” refers to steroids and/or lipids containing steroidal moieties (e.g., sterols and/or lipids containing sterol moieties). Incorporation of structural lipids in the lipid nanoparticle can help mitigate aggregation of other lipids in the lipid nanoparticle.

Structural lipids can include, but are not limited to, sterols (e.g., phytosterols or zoosterols). Sterols can include, but are not limited to, cholesterol, beta-sitosterol, fecosterol, ergosterol, sitosterol, campesterol, stigmasterol, brassicasterol, ergosterol, tomatidine, tomatine, ursolic acid, or alpha-tocopherol.

Non-Cationic Helper Lipids

The lipid nanoparticles of the invention can include one or more non-cationic helper lipids (e.g., a phospholipid). For example, lipid nanoparticles can include a non-cationic helper lipid or one or more non-cationic helper lipids (e.g., two or more non-cationic helper lipids, three or more non-cationic helper lipids, four or more non-cationic helper lipids, or five or more non-cationic helper lipids). The non-cationic helper lipids described herein may be advantageously used in lipid nanoparticles of the invention for the delivery of nucleic acid molecules to a cell (e.g., mammalian cell).

Non-cationic helper lipids include, but are not limited to, phospholipids (e.g., polyunsaturated phospholipids) and fatty acids (e.g., oleic acid).

Phospholipids include a phospholipid moiety and one or more fatty acid moieties. For example, a phospholipid may be a lipid according to the formula:

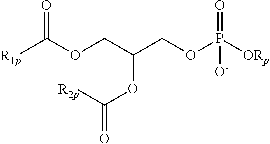

in which Rₚ represents a phospholipid moiety and R₁ₚ and R₂ₚ represent fatty acid moieties with or without saturation that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated.

Phospholipids include, but are not limited to, 1,2-distearoyl-sn-glycero-3-phosphocholine (DSPC), 1,2-dioleoyl-sn-glycero-3-phosphoethanolamine (DOPE), or both DSPC and DOPE. Phospholipids useful in the compositions and methods of the invention may be selected from the non-limiting group consisting of DSPC, DOPE, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

Fatty acids include, but are not limited to, short-chain fatty acids (SOFA), medium-chain fatty acids (MCFA), long-chain fatty acids (LCFA), or very long-chain fatty acids (VLCFA).

Short-chain fatty acids include, but are not limited to, butyric acid, isobutyric acid, valeric acid, and isovaleric acid. Medium-chain fatty acids include, but are not limited to, caproic acid, caprylic acid, capric acid, and lauric acid. Long-chain fatty acids include, but are not limited to, pentadecylic acid, palmitic acid, margaric acid, stearic acid, nonadecylic acid, arachidic acid, heneicosylic acid, behenic acid, palmitoleic acid, oleic acid, elaidic acid, gondoic acid, erucic acid, sapienic acid, paullinic acid, myristic acid, myristoleic acid, vaccenic acid, eicosapentaenoic acid, erucic acid, linolelaidic acid, docsahexaenoic acid, myristic acid, or linoleic acid. Very long-chain fatty acids include, but are not limited to, tricosylic acid, lignoceric acid, cerotic acid, nervonic acid, pentacosylic acid, heptacosylic acid, montanic acid, nonacosylic acid, melissic acid, or henatriacontylic acid.

PEG-Lipids

The lipid nanoparticles of the invention can include one or more PEG-lipids. For example, lipid nanoparticles can include a PEG-lipid or one or more PEG-lipids (e.g., two or more PEG-lipids, three or more PEG-lipids, four or more PEG-lipids, or five or more PEG-lipids). The PEG-lipids described herein may be advantageously used in lipid nanoparticles of the invention for the delivery of nucleic acid molecules to a cell (e.g., mammalian cell).

PEG-lipids can be PEG-modified phosphatidylethanolamines, PEG-modified phosphatidic acids,

PEG-modified ceramides, PEG-ceramide conjugates, PEG-modified dialkylamines, PEG-modified diacylglycerols, PEG-modified 1,2-diacyloxypropan-3-amines, and PEG-modified dialkylglycerols. PEG-lipids include, but are not limited to, 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), PEG-1,2-dimyristyloxlpropyl-3-amine (PEG-c-DMA), R-3-[(ω-methoxy poly(ethylene glycol)₂₀₀₀)carbamoyl)]-1,2-dimyristyloxlpropyl-3-amine (PEG-c-DOMG), PEG-1,2-dilauroyl-sn-glycero-3-phosphoethanolamine (PEG-DLPE), PEG-1,2-dimyristoyl-sn-glycero-3-phosphoethanolamine (PEG-DMPE), PEG-1,2-dipalmitoyl-sn-glycero-3-phosphocholine (PEG-DPPC), 1-O-(2′-(ω-methoxy-polyethylene-glycol)succinoyl)-2-N-myristoyl-sphingosine (PEG-CerC14), or 1-O-(2′-(ω-methoxy-polyethylene-glycol)succinoyl)-2-N-arachidoyl-sphingosine (PEG-CerC20).

The aliphatic chains of the PEG-lipids can each have 14 to 22 carbons (e.g., 14 to 16, 16 to 18, 14 to 20, or 14 to 18 carbons). In some embodiments, a PEG moiety, for example an mPEG-NH2, has a size of about 1000, 2000, 5000, 10,000, 15,000 or 20,000 daltons. In some embodiments, the PEG-lipid is PEG2k-DMG.

The lipid nanoparticles described herein can include a PEG-lipid which is a non-diffusible PEG. Non-limiting examples of non-diffusible PEGs include PEG-DSG and PEG-DSPE.

PEG-lipids can include those described in U.S. Pat. No. 8,158,601 and International Publication No. WO 2015/130584 and WO 2012/099755. The PEG-lipids described herein can be synthesized as described in International Patent Application No. PCT/US2016/000129.

In some embodiments, the PEG-lipid is a modified form of PEG-DMG. PEG-DMG has the following structure:

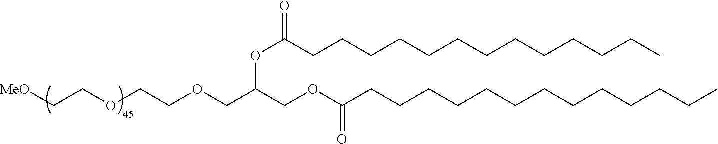

In certain embodiments, a PEG lipid useful in the present invention is a PEGylated fatty acid.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

Other Components

A composition of the invention may include one or more components in addition to those described in the preceding sections. For example, a composition may include one or more small hydrophobic molecules such as a vitamin (e.g., vitamin A or vitamin E) or a sterol.

Compositions may also include one or more permeability enhancer molecules, carbohydrates, polymers, therapeutic agents, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate, polyvinylpyrrolidone.

Therapeutic agents may include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a composition (e.g., by coating, adsorption, covalent linkage, or other process).

In addition to these components, compositions of the invention may include any substance useful in pharmaceutical compositions. For example, the composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Alcohol preservatives include, but are not limited to, ethanol, polyethylene glycol, phenol, benzyl alcohol, phenolic compounds, bisphenol, chlorobutanol, hydroxybenzoate, and/or phenylethyl alcohol. Acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

RNA

An RNA may be a messenger RNA (mRNA). An mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” should be understood to include the canonical guanosine triphosphate, 7-methylguanosine triphosphate, or any other definition encompassed herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine.

In some embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. Cap species include m⁷GpppG, m⁷Gpppm⁷G, m⁷3′dGpppG, m₂^{7,O3′}GpppG, m₂^{7,O3′}GppppG, m₂^{7,O2′}GppppG m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

An mRNA may instead or additionally include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

Compositions

A lipid nanoparticle of the invention includes an ionizable lipid and a β-sitosterol, campesterol, and/or sitostanol purified by any of the foregoing methods described in this invention. The lipid nanoparticle can further include one or more structural lipids, one or more non-cationic helper lipids, one or more PEG-lipids, or any combination thereof. For example, a lipid nanoparticle can include 40 mol % of ionizable lipid, about 15 mol % non-cationic helper lipid, about 43.5 mol % β-sitosterol, campesterol, and/or sitostanol purified by any of the foregoing methods described in this invention, and about 1.5% PEG-lipid. The lipid nanoparticle can further include a nucleic acid molecule (e.g., mRNA).

A composition of the invention may be designed for one or more specific applications or targets. For example, a composition may be designed to deliver mRNA to a particular cell, tissue, organ, or system or group thereof in a mammal's body, such as the renal system. Physiochemical properties of compositions may be altered in order to increase selectivity for particular bodily targets. For instance, particle sizes may be adjusted based on the fenestration sizes of different organs. The mRNA included in a composition may also depend on the desired delivery target or targets. For example, an mRNA may be selected for a particular indication, condition, disease, or disorder and/or for delivery to a particular cell, tissue, organ, or system or group thereof (e.g., localized or specific delivery). A composition may include one or more mRNA molecules encoding one or more polypeptides of interest.

The amount of mRNA in a composition may depend on the size, sequence, and other characteristics of the mRNA. The amount of mRNA in a composition may also depend on the size, composition, desired target, and other characteristics of the composition. The relative amounts of mRNA and other elements (e.g., lipids) may also vary. In some embodiments, the wt/wt ratio of one or more ionizable lipids, β-sitosterol, campesterol, and/or sitostanol purified by any of the foregoing methods described in this invention, one or more non-cationic helper lipids, one or more PEG-lipids, or any combination thereof to an mRNA in a composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of one or more ionizable lipids, β-sitosterol, campesterol, and/or sitostanol purified by any of the foregoing methods described in this invention, one or more non-cationic helper lipids, one or more PEG-lipids, or any combination thereof to an mRNA may be from about 10:1 to about 40:1. The amount of mRNA in a composition may, for example, be measured using absorption spectroscopy (e.g., ultraviolet-visible spectroscopy).

In some embodiments, mRNA, lipid nanoparticles, and amounts thereof may be selected to provide a specific N:P ratio. The N:P ratio of the composition refers to the molar ratio of nitrogen atoms in one or more lipids to the number of phosphate groups in an mRNA. In general, a lower N:P ratio is preferred. The mRNA, lipid nanoparticles, and amounts thereof may be selected to provide an N:P ratio from about 2:1 to about 8:1, such as 2:1, 3:1, 4:1, 5:1, 6:1, 7:1, and 8:1. In certain embodiments, the N:P ratio may be from about 2:1 to about 5:1.

Physical Properties

The characteristics of a composition may depend on the components thereof. Similarly, the characteristics of a composition may depend on the absolute or relative amounts of its components. For instance, a composition including a higher molar fraction of a cationic lipid may have different characteristics than a composition including a lower molar fraction of a cationic lipid. Characteristics may also vary depending on the method and conditions of preparation of the composition.

Compositions may be characterized by a variety of methods. For example, microscopy (e.g., transmission electron microscopy or scanning electron microscopy) may be used to examine the morphology and size distribution of a composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a composition, such as particle size, polydispersity index, and zeta potential.

The mean size of a composition of the invention may be between 10 s of nm and 100 s of nm. For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a composition may be from about 80 nm to about 120 nm. In a particular embodiment, the mean size may be about 90 nm.

A composition of the invention may be relatively homogenous. A polydispersity index may be used to indicate the homogeneity of a composition, e.g., the particle size distribution of the compositions. A small (e.g., less than 0.3) polydispersity index generally indicates a narrow particle size distribution. A composition of the invention may have a polydispersity index from about 0 to about 0.18, such as 0.01, 0.02, 0.03, 0.04, 0.05, 0.06, 0.07, 0.08, 0.09, 0.10, 0.11, 0.12, 0.13, 0.14, 0.15, 0.16, 0.17, or 0.18. In some embodiments, the polydispersity index of a composition may be from about 0.13 to about 0.17.

The zeta potential of a composition may be used to indicate the electrokinetic potential of the composition. For example, the zeta potential may describe the surface charge of a composition. Compositions with relatively low charges, positive or negative, are generally desirable, as more highly charged species may interact undesirably with cells, tissues, and other elements in the body. In some embodiments, the zeta potential of a composition of the invention may be from about −10 mV to about +20 mV.

The efficiency of encapsulation of an mRNA describes the amount of mRNA that is encapsulated or otherwise associated with a composition after preparation, relative to the initial amount provided. The encapsulation efficiency is desirably high (e.g., close to 100%). The encapsulation efficiency may be measured, for example, by comparing the amount of mRNA in a solution containing the composition before and after breaking up the composition with one or more organic solvents or detergents. Fluorescence may be used to measure the amount of free mRNA in a solution. For the compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A composition of the invention may optionally comprise one or more coatings. For example, a composition may be formulated in a capsule, film, or tablet having a coating. A capsule, film, or tablet including a composition of the invention may have any useful size, tensile strength, hardness, or density.

Pharmaceutical Compositions

A lipid nanoparticle the invention may be formulated in whole or in part as pharmaceutical compositions. Pharmaceutical compositions of the invention may include one or more compositions. For example, a pharmaceutical composition may include one or more compositions including one or more different mRNAs. Pharmaceutical compositions of the invention may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition of the invention, except insofar as any conventional excipient or accessory ingredient may be incompatible with one or more components of a composition of the invention. An excipient or accessory ingredient may be incompatible with a component of a composition if its combination with the component may result in any undesirable biological effect or otherwise deleterious effect.

In some embodiments, one or more excipients or accessory ingredients may make up greater than 50% of the total mass or volume of a pharmaceutical composition including a composition of the invention. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more compositions.

Compositions and/or pharmaceutical compositions including one or more compositions may be administered to any patient or subject, including those patients or subjects that may benefit from a therapeutic effect provided by the delivery of an mRNA to one or more particular cells, tissues, organs, or systems or groups thereof, such as the renal system. Although the descriptions provided herein of compositions and pharmaceutical compositions including compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., composition). The amount of the active ingredient is generally equal to the dosage of the active ingredient which would be administered to a subject and/or a convenient fraction of such a dosage such as, for example, one-half or one-third of such a dosage.

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay, silicates), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Embedding compositions which can be used include, but are not limited to, polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### EXAMPLES

### Example 1. Esterification of Sterol—Method A

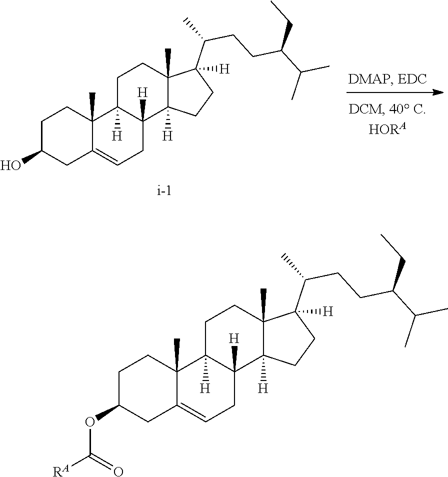

To a solution of sterol i-1 (200 mg, 0.482 mmol, 1 equiv.) in dichloromethane (DCM, 2 mL) was added carboxylic acid R^{A}COOH (1.1 equiv.). To this was added 4-(dimethylamino)pyridine (DMAP, 11.8 mg, 0.096 mmol, 0.2 equiv.) and 1-ethyl-3-(3-dimethylaminopropyl)carbodiimide (EDC, 120.8 mg, 0.63 mmol, 1.5 equiv.). The reaction was stirred at 40° C. and monitored by TLC and/or LC/MS. After complete conversion of starting material, the reaction was cooled to room temperature, optionally diluted with DCM, and quenched with aqueous NaHCO₃ and aqueous NaCl. The organic layer was dried over anhydrous sodium sulfate and concentrated to give the desired product.

Compounds 1-3, 5-9, 11-16, and 18-19 were synthesized using the protocol described in Method A above and the appropriate carboxylic acid (R^{A}COOH) as shown in Table 2.

TABLE 2

| Product | R^{A}COOH | Yield | Appearance |
|---|---|---|---|
| Compound 1 | Stearic acid | 99% | Off-white solid |
| Compound 2 | Trifluoroacetic acid | 99% | Yellow solid |
| Compound 3 | Cyclopropanecarboxylic acid | 52% | Off-white solid |
| Compound 5 | Isobutyric Acid | 80% | Brown solid |
| Compound 6 | Benzoic acid | 98% | Off-white solid |
| Compound 7 | p-Toluic Acid | 87% | Off-white solid |
| Compound 8 | 2-(methylthio)acetic acid | 87% | Brown solid |
| Compound 9 | 2-(methylsulfonyl)acetic acid | 69% | Off-white solid |
| Compound 11 | 3-(pyridine-4-yl)propanoic acid | 65% | Light pink solid |
| Compound 12 | 3-morpholinopropanoic acid | 70% | Off-white solid |
| Compound 13 | 3-(4-methylpiperazin-1-yl)propanoic acid | 89% | Yellow oil |
| Compound 14 | 3-furoic acid | 76% | Tan solid |
| Compound 15 | 3-thiophenecarboxylic acid | 75% | Brown solid |
| Compound 16 | Terephthalic acid | 22% | Pale yellow solid |
| Compound 18 | (3-carboxypropyl)trimethylammonium chloride | 46% | Off-white solid |
| Compound 19 | N,N-Dimethylglycine HCl | 91% | Yellow solid |

### Example 2. Esterification of Sterol—Method B

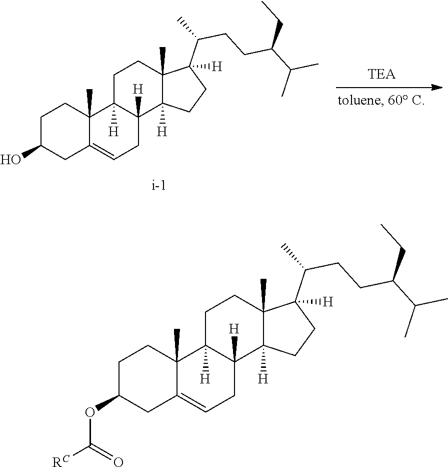

To a solution of sterol i-1 (200 mg, 0.482 mmol, 1 equiv.) in toluene (2 mL) was added anhydride RB (1.66 equiv.). To this was added triethylamine (TEA, 16.9 μL, 0.121 mmol, 0.25 equiv.). The reaction was stirred at 60° C. and monitored by LC/MS. After complete conversion of starting material, the reaction was cooled to room temperature, optionally diluted with DCM, quenched with 3 mL of water, and extracted with DCM (3×) The organic layer was washed with 10 mL of 2N HCl (3×) and then with 10 mL of water (3×). The organic layer was then washed with 10 mL of brine, dried over anhydrous sodium sulfate, and concentrated to give the desired product.

Compounds 31-33 were synthesized using the protocol described in Method B above and the appropriate anhydride (RB) as shown in Table 3.

TABLE 3

| Product | R^{B} | Yield | Appearance |
|---|---|---|---|
| Compound 31 | Succinic Anhydride | 89% | Off-white solid |
| Compound 32 | Diglycolic Anhydride | 77% | Brown solid |
| Compound 33 | Phthalic Anhydride | >95% | Pink solid |

### Example 3. Hydrolysis of Sterol Ester

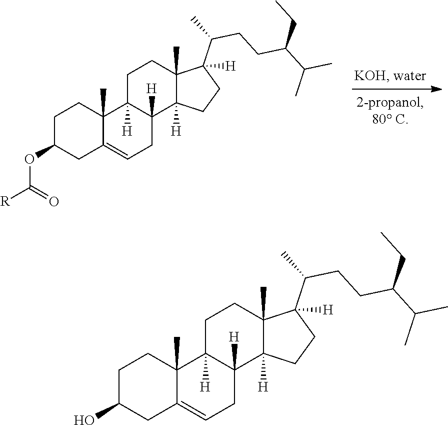

To a solution of sterol ester i-2 (10.6 g, 18.83 mmol, 1 equiv.) in 2-propanol (100 mL) was added potassium hydroxide (2.2 g, 39.55 mmol, 2.1 equiv.) and water (100 mL). The reaction mixture was heated to 80° C. and monitored by LC/MS. After complete conversion of starting material, the reaction was cooled to room temperature, optionally diluted with 300 mL of DCM, quenched with 200 mL of 5% brine solution. The organic layer was washed with 300 mL of 5% sodium bicarbonate solution (3×) and concentrated to give the desired product.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A compound having the structure of Formula I: 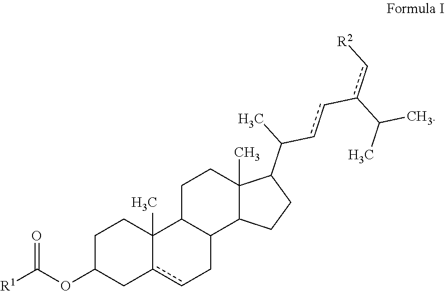
wherein the dotted lines represent optional double bonds;
R¹ is trifluoromethyl, trichloromethyl, iso-propyl, tert-butyl, 4-methyl-phenyl, 4-carboxylic acid-phenyl, 3-carboxylic acid-propyl, optionally substituted C₂-C₉ heteroaryl, optionally substituted C₁-C₆ alkyl C₂-C₉ heteroaryl, optionally substituted C₁-C₆ alkyl C₂-C₉ heterocyclyl, optionally substituted C₁-C₆ heteroalkyl, optionally substituted C₃-C₈ cycloalkyl, optionally substituted C₆-C₂₁ alkyl, or optionally substituted C₁-C₂₁ alkenyl; and
R² is hydrogen or methyl;
or a pharmaceutically acceptable salt thereof.

2. The compound of claim 1, wherein the compound has the structure:

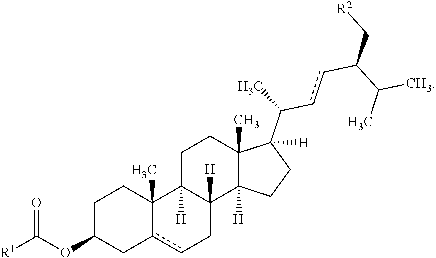

3. The compound of claim 1, wherein the compound has the structure:

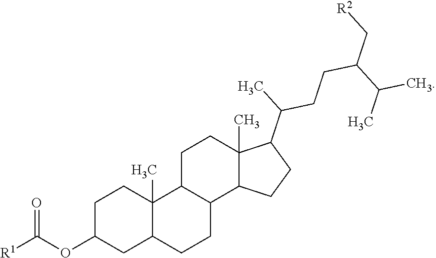

4. The compound of claim 1, wherein the compound has the structure:

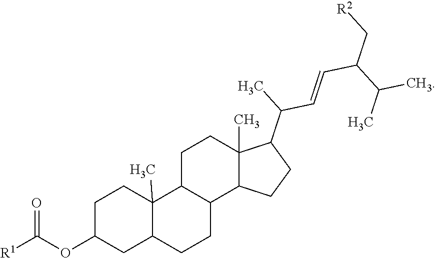

5. The compound of claim 1, wherein the compound has the structure:

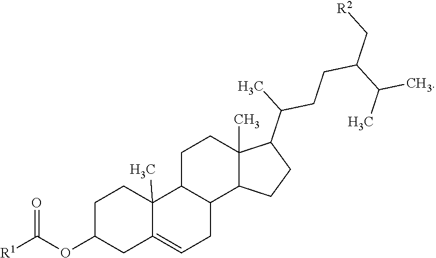

6. The compound of claim 1, wherein the compound has the structure:

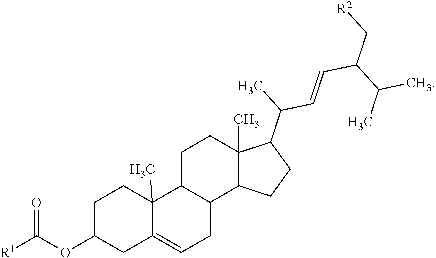

7. The compound of claim 1, wherein the compound has the structure:

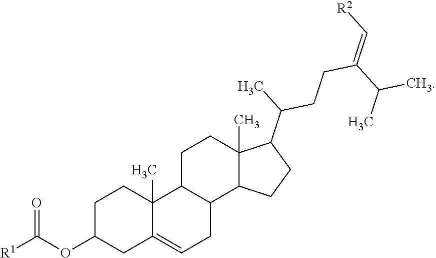

8. The compound of claim 1, wherein the compound has the structure:

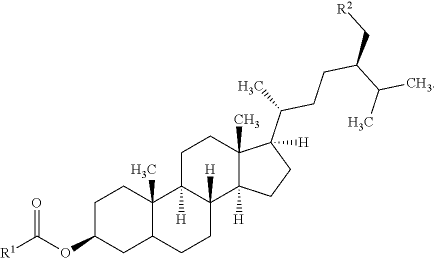

9. The compound of claim 1, wherein the compound has the structure:

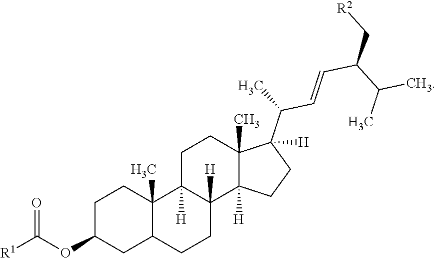

10. The compound of claim 1, wherein the compound has the structure:

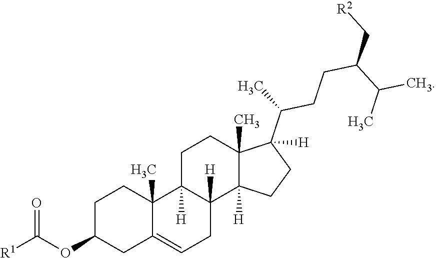

11. The compound of claim 1, wherein the compound has the structure:

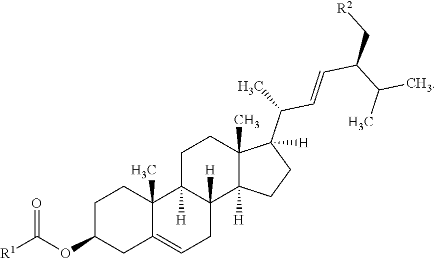

12. The compound of any one of claims 1 to 11, wherein R² is hydrogen.

13. The compound of any one of claims 1 to 11, wherein R² is methyl.

14. The compound of any one of claims 1 to 13, wherein R¹ is —(CH₂)₁₆CH₃, —CF₃, —CCl₃, cyclopropyl, tert-butyl, iso-propyl, 4-methyl-phenyl,

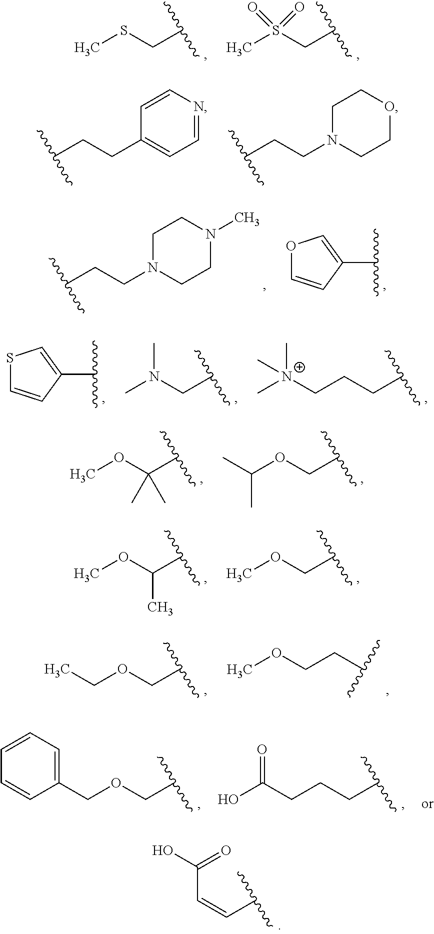

15. A composition comprising a compound of any one of claims 1 to 14 and an excipient.

16. A method of producing purified β-sitosterol, the method comprising:
(a) obtaining a sample comprising β-sitosterol and one or more other sterols;
(b) reacting the sample under conditions sufficient to produce a β-sitosterol ester;
(c) separating the β-sitosterol ester from the one or more other sterols in the sample to produce a sample of β-sitosterol ester; and
(d) reacting the sample of β-sitosterol ester under conditions sufficient to hydrolyze the β-sitosterol ester,
thereby producing purified β-sitosterol.

17. The method of claim 16, wherein the method further comprises: (e) recrystallizing the product produced in step (d).

18. The method of claim 16 or 17, wherein the β-sitosterol ester has the structure of Formula II: 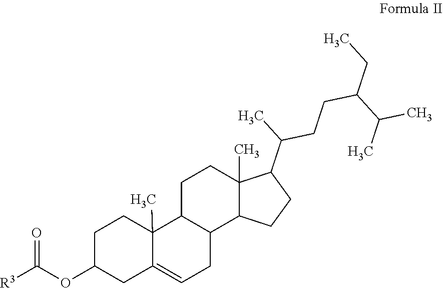
wherein R³ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

19. The method of claim 18, wherein R³ is —(CH₂)₁₆CH₃, —CF₃, —CCl₃, cyclopropyl, tert-butyl, iso-propyl, phenyl, 4-methyl-phenyl,

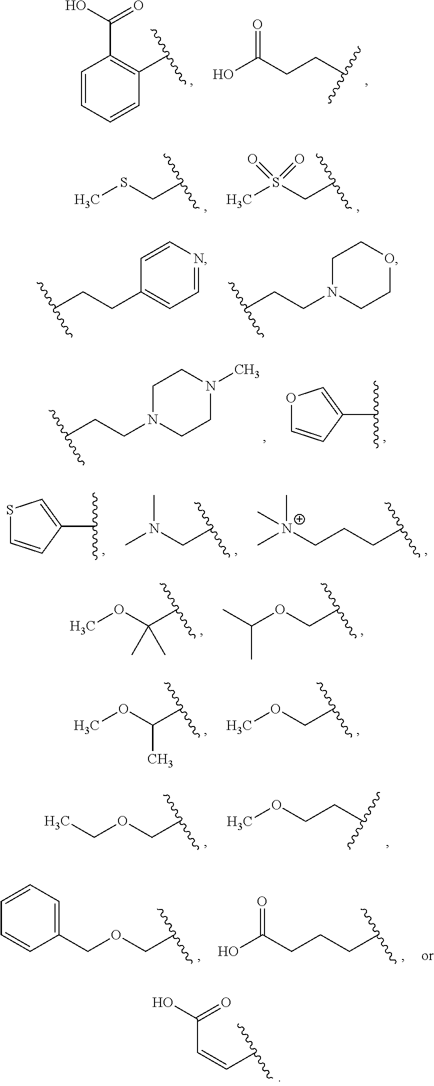

20. A method of producing purified campesterol, the method comprising:
(a) obtaining a sample comprising campesterol and one or more other sterols;
(b) reacting the sample under conditions sufficient to produce a campesterol ester;
(c) substantially separating the campesterol ester from the one or more other sterols in the sample to produce a sample of campesterol ester; and
(d) reacting the sample of campesterol ester under conditions sufficient to hydrolyze the campesterol ester,
thereby producing purified campesterol.

21. The method of claim 20, wherein the method further comprises: (e) recrystallizing the product produced in step (d).

22. The method of claim 20 or 21, wherein the campesterol ester has the structure of Formula III: 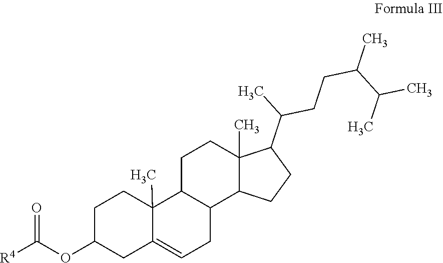
wherein R⁴ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

23. The method of claim 22, wherein R⁴ is —(CH₂)₁₆CH₃, —CF₃, —CCl₃, cyclopropyl, tert-butyl, iso-propyl, phenyl, 4-methyl-phenyl,

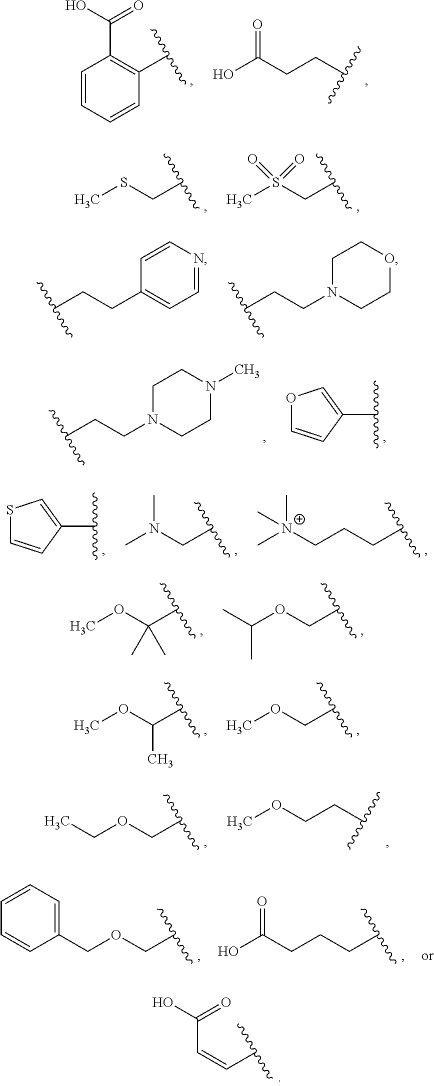

24. A method of producing purified sitostanol, the method comprising:
(a) obtaining a sample comprising sitostanol and one or more other sterols;
(b) reacting the sample under conditions sufficient to produce a sitostanol ester;
(c) separating the sitostanol ester from the one or more other sterols in the sample to produce a sample of sitostanol ester; and
(d) reacting the sample of sitostanol ester under conditions sufficient to hydrolyze the sitostanol ester,
thereby producing purified sitostanol.

25. The method of claim 24, wherein the method further comprises: (e) recrystallizing the product produced in step (d).

26. The method of claim 24 or 25, wherein the sitostanol ester has the structure of Formula IV: 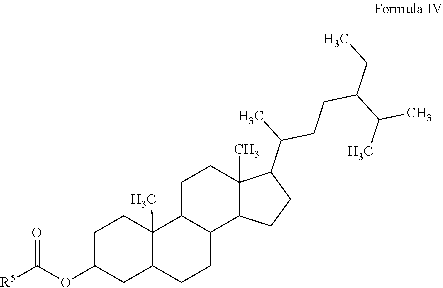
wherein R⁵ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

27. The method of claim 26, wherein R⁵ is —(CH₂)₁₆CH₃, —CF₃, —CCl₃, cyclopropyl, tert-butyl, iso-propyl, phenyl, 4-methyl-phenyl,

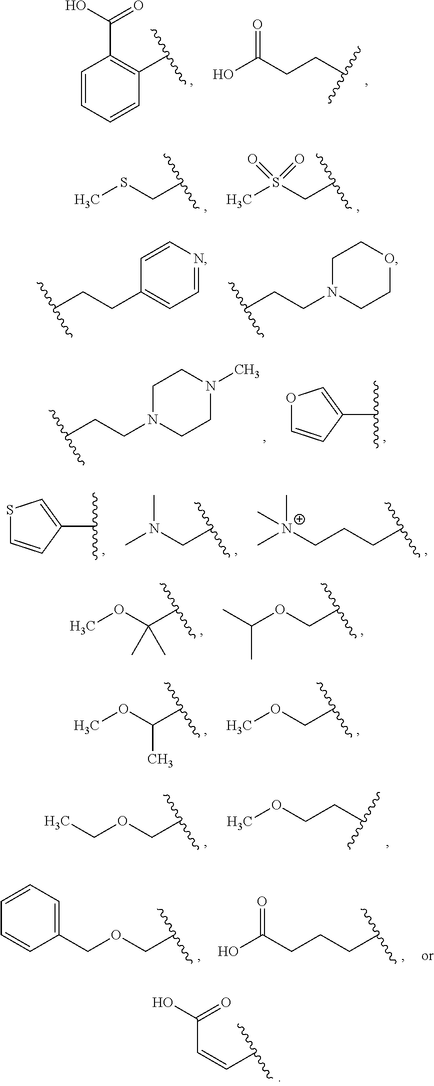

28. A method of producing purified stigmasterol, the method including:
(a) obtaining a sample including stigmasterol and one or more other sterols;
(b) reacting the sample under conditions sufficient to produce a stigmasterol ester;
(c) separating the stigmasterol ester from the one or more other sterols in the sample to produce a sample of stigmasterol ester; and
(d) reacting the sample of stigmasterol ester under conditions sufficient to hydrolyze the stigmasterol ester,
thereby producing purified stigmasterol.

29. The method of claim 28, wherein the method further includes: (e) recrystallizing the product produced in step (d).

30. The method of claim 28 or 29, wherein the stigmasterol ester has the structure of Formula V: 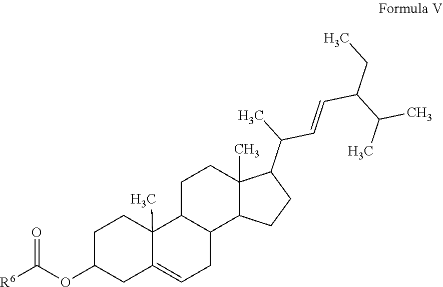
wherein R⁶ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

31. The method of claim 30, wherein R⁶ is —(CH₂)₁₆CH₃, —CF₃, —CCl₃, cyclopropyl, tert-butyl, iso-propyl, phenyl, 4-methyl-phenyl,

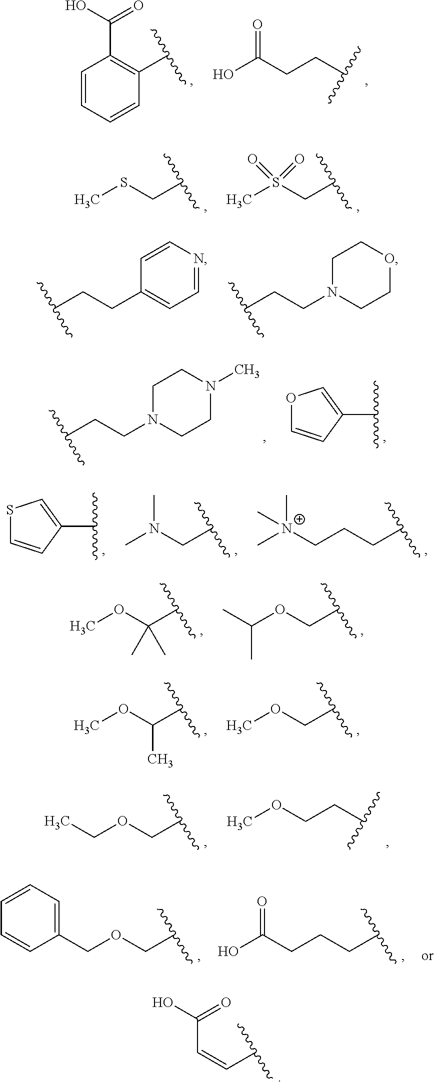

32. A method of producing purified campestanol, the method including:
(a) obtaining a sample including campestanol and one or more other sterols;
(b) reacting the sample under conditions sufficient to produce a campestanol ester;
(c) separating the campestanol ester from the one or more other sterols in the sample to produce a sample of campestanol ester; and
(d) reacting the sample of campestanol ester under conditions sufficient to hydrolyze the campestanol ester,
thereby producing purified campestanol.

33. The method of claim 32, wherein the method further includes: (e) recrystallizing the product produced in step (d).

34. The method of claim 32 or 33, wherein the campestanol ester has the structure of Formula VI: 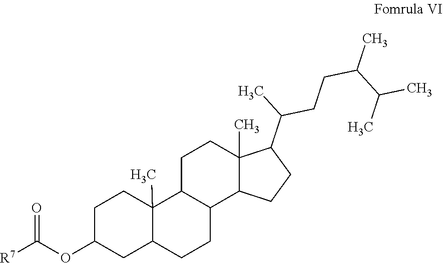
wherein R⁷ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

35. The method of claim 34, wherein R⁷ is —(CH₂)₁₆CH₃, —CF3, —CCl₃, cyclopropyl, tert-butyl, iso-propyl, phenyl, 4-methyl-phenyl,

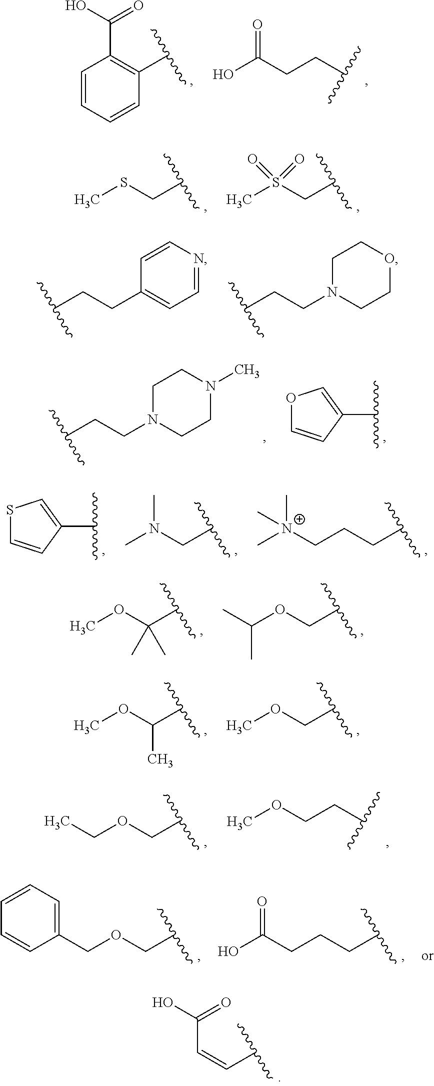

36. A method of producing purified brassicasterol, the method including:
(a) obtaining a sample including brassicasterol and one or more other sterols;
(b) reacting the sample under conditions sufficient to produce a brassicasterol ester;
(c) separating the brassicasterol ester from the one or more other sterols in the sample to produce a sample of brassicasterol ester; and
(d) reacting the sample of brassicasterol ester under conditions sufficient to hydrolyze the brassicasterol ester,
thereby producing purified brassicasterol.

37. The method of claim 36, wherein the method further includes: (e) recrystallizing the product produced in step (d).

38. The method of claim 36 or 37, wherein the brassicasterol ester has the structure of Formula VII: 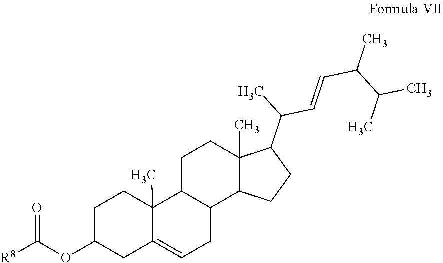
wherein R⁸ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

39. The method of claim 38, wherein R⁸ is —(CH₂)₁₆CH₃, —CF3, —CCl₃, cyclopropyl, tert-butyl, iso-propyl, phenyl, 4-methyl-phenyl,

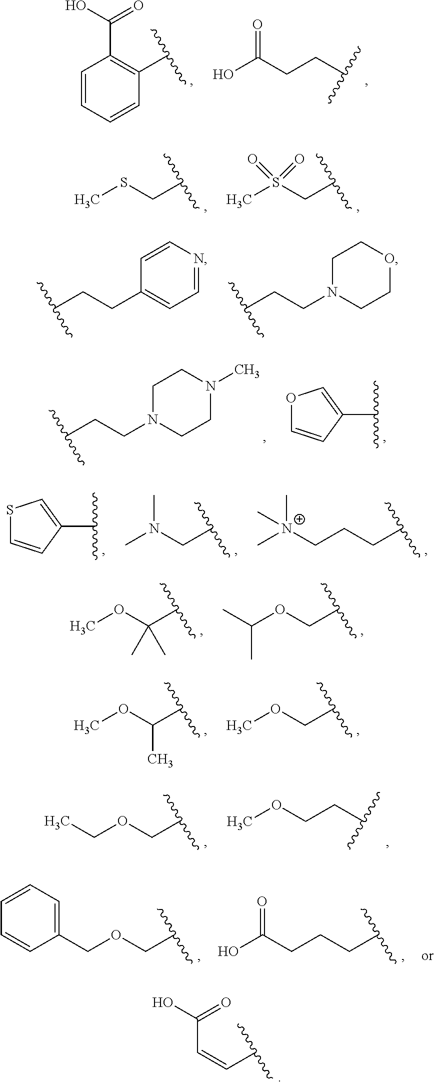

40. A method of producing purified fucosterol, the method including:
(a) obtaining a sample including fucosterol and one or more other sterols;
(b) reacting the sample under conditions sufficient to produce a fucosterol ester;
(c) separating the fucosterol ester from the one or more other sterols in the sample to produce a sample of fucosterol ester; and
(d) reacting the sample of fucosterol ester under conditions sufficient to hydrolyze the fucosterol ester,
thereby producing purified fucosterol.

41. The method of claim 40, wherein the method further includes: (e) recrystallizing the product produced in step (d).

42. The method of claim 40 or 41, wherein the fucosterol ester has the structure of Formula VIII: 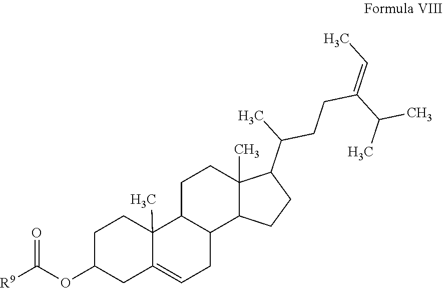
wherein R⁹ is optionally substituted C₂-C₉ heteroaryl, optionally substituted C₁-C₆ alkyl C₂-C₉ heteroaryl, optionally substituted C₆-C₁₀ aryl, optionally substituted C₁-C₆ alkyl C₆-C₁₀ aryl, optionally substituted C₁-C₆ alkyl C₂-C₉ heterocyclyl, optionally substituted C₁-C₆ heteroalkyl, optionally substituted C₃-C₈ cycloalkyl, optionally substituted C₁-C₂₁ alkyl, or optionally substituted C₁-C₂₁ alkenyl,
or a pharmaceutically acceptable salt thereof.

43. The method of claim 42, wherein R⁹ is —(CH₂)₁₆CH₃, —CF₃, —CCl₃, cyclopropyl, tert-butyl, iso-propyl, phenyl, 4-methyl-phenyl,

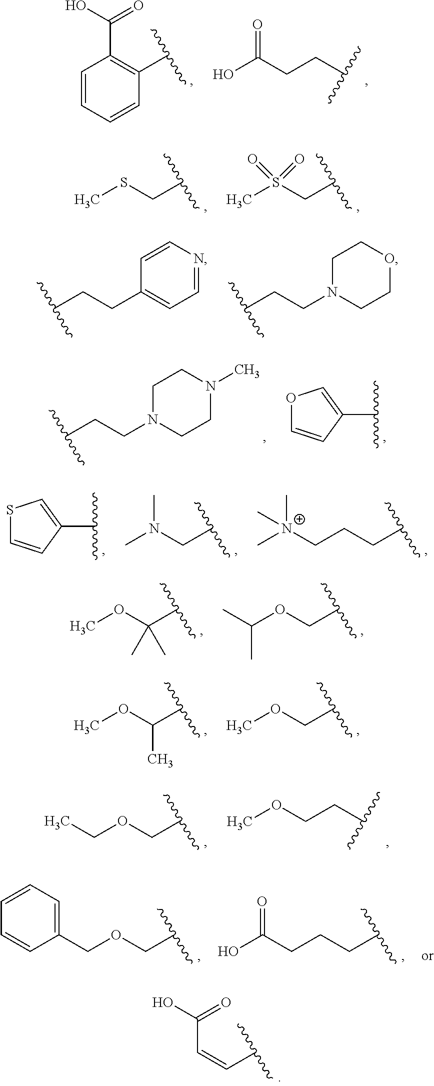

44. The method of any one of claims 16 to 43, wherein the conditions sufficient to produce a β-sitosterol ester, campesterol ester, sitostanol ester, stigmasterol ester, campestanol ester, fucosterol ester, or brassicasterol ester comprise (i) an acid anhydride and a base; (ii) a carboxylic acid and a carboxyl activating agent; or (iii) an acyl chloride.

45. The method of claim 44, wherein the conditions sufficient to produce a β-sitosterol ester, campesterol ester, sitostanol ester, stigmasterol ester, campestanol ester, fucosterol ester, or brassicasterol ester comprise an acid anhydride and a base.

46. The method of claim 45, wherein the base is an organic amine base.

47. The method of claim 46, wherein the organic amine base is triethylamine.

48. The method of claim 44, wherein the conditions sufficient to produce β-sitosterol ester, campesterol ester, sitostanol ester, stigmasterol ester, campestanol ester, fucosterol ester, or brassicasterol ester comprise a carboxylic acid and a carboxyl activating agent.

49. The method of claim 48, wherein the carboxyl activating agent is 1-Ethyl-3-(3-dimethylaminopropyl)carbodiimide.

50. The method of claim 44, wherein the conditions sufficient to produce a β-sitosterol ester, campesterol ester, sitostanol ester, stigmasterol ester, campestanol ester, fucosterol ester, or brassicasterol ester comprise an acyl chloride.

51. The method of any one of claims 44 to 50, wherein the conditions sufficient to produce a β-sitosterol ester, campesterol ester, or sitostanol ester further comprise 4-(dimethylamino)pyridine.

52. The method of any one of claims 16 to 51, wherein the separating comprises normal phase purification.

53. The method of any one of claims 16 to 51, wherein the separating comprises reverse phase purification.

54. The method of any one of claims 16 to 53, wherein the conditions sufficient to hydrolyze the β-sitosterol ester, campesterol ester, sitostanol ester, stigmasterol ester, campestanol ester, fucosterol ester, or brassicasterol ester comprise water, an alcohol and a base.

55. The method of any one of claims 16 to 54, wherein the purified sample of β-sitosterol ester, campesterol ester, sitostanol ester, stigmasterol ester, campestanol ester, fucosterol ester, or brassicasterol ester has a purity of at least 90%.

56. The method of claim 55, wherein the purified sample of β-sitosterol ester, campesterol ester, sitostanol ester, stigmasterol ester, campestanol ester, fucosterol ester, or brassicasterol ester has a purity of at least 95%.

57. The method of claim 56, wherein the purified sample of β-sitosterol ester, campesterol ester, sitostanol ester, stigmasterol ester, campestanol ester, fucosterol ester, or brassicasterol ester has a purity of at least 99%.

58. A method of producing a lipid nanoparticle, the method comprising
(i) preparing purified β-sitosterol, campesterol, sitostanol, stigmasterol, campestanol, fucosterol, and/or brassicasterol by the method of any one of claims 16 to 57; and
(ii) contacting the purified β-sitosterol, campesterol, sitostanol, stigmasterol, campestanol, fucosterol, and/or brassicasterol and an ionizable lipid under conditions sufficient to form a lipid nanoparticle,
thereby producing a lipid nanoparticle.

59. The method of claim 58, wherein the method further comprises contacting the β-sitosterol, campesterol, sitostanol, stigmasterol, campestanol, fucosterol, and/or brassicasterol and the ionizable lipid with a non-ionizable helper lipid and/or a PEG-lipid.

60. The method of claim 58 or 59, wherein the method further comprises contacting the lipid nanoparticle with an mRNA encoding a polypeptide under conditions sufficient for the lipid nanoparticle to encapsulate the mRNA.

61. A composition comprising two or more sterols, wherein the two or more sterols comprise β-sitosterol and campesterol, wherein β-sitosterol comprises 95-99.9% of the sterols in the composition and campesterol comprises 0.1-5% of the sterols in the composition.

62. The composition of claim 61, wherein the composition further comprises sitostanol.

63. The composition of claim 62, wherein β-sitosterol comprises 95-99.9%, campesterol comprises 0.05-4.95%, and sitostanol comprises 0.05-4.95% of the sterols in the composition.

64. A composition comprising two or more sterols, wherein the two or more sterols comprise β-sitosterol and sitostanol, wherein β-sitosterol comprises 95-99.9% of the sterols in the composition and sitostanol comprises 0.1-5% of the sterols in the composition.

65. The composition of claim 64, wherein the composition further comprises campesterol.

66. The composition of claim 65, wherein β-sitosterol comprises 95-99.9%, campesterol comprises 0.05-4.95%, and sitostanol comprises 0.05-4.95% of the sterols in the composition.

67. A composition comprising a plurality of lipid nanoparticles, wherein the plurality of lipid nanoparticles comprise an ionizable lipid and two or more sterols, wherein the two or more sterols comprise β-sitosterol, and campesterol and β-sitosterol comprises 95-99.9% of the sterols in the composition and campesterol comprises 0.1-5% of the sterols in the composition.

68. The composition of claim 67, wherein the two or more sterols further comprises sitostanol.

69. The composition of claim 68, wherein β-sitosterol comprises 95-99.9%, campesterol comprises 0.05-4.95%, and sitostanol comprises 0.05-4.95% of the sterols in the composition.

70. A composition comprising a plurality of lipid nanoparticles, wherein the plurality of lipid nanoparticles comprise an ionizable lipid and two or more sterols, wherein the two or more sterols comprise β-sitosterol, and sitostanol and β-sitosterol comprises 95-99.9% of the sterols in the composition and sitostanol comprises 0.1-5% of the sterols in the composition.

71. The composition of claim 70, wherein the two or more sterols further comprises campesterol.

72. The composition of claim 71, wherein β-sitosterol comprises 95-99.9%, campesterol comprises 0.05-4.95%, and sitostanol comprises 0.05-4.95% of the sterols in the composition.

73. The composition of any one of claims 70 to 72, wherein the plurality of lipid nanoparticles further comprise a non-ionizable helper lipid and/or a PEG-lipid.

74. A lipid nanoparticle comprising:
(i) an ionizable lipid; and
(ii) a structural component,
wherein the structural component comprises a compound of any one of claims 1 to 14.

75. The lipid nanoparticle of claim 74, wherein the lipid nanoparticle further comprises a nucleic acid molecule.

76. A lipid nanoparticle comprising:
(i) an ionizable lipid;
(ii) a structural component;
(iii) optionally, a non-cationic helper lipid;
(iv) optionally, a PEG-lipid; and
(v) a nucleic acid molecule,
wherein the structural component comprises a compound of any one of claims 1 to 14 and optionally a structural lipid.

77. The lipid nanoparticle of any one of claims 74 to 76, wherein the lipid nanoparticle comprises the compound of any one of claims 1 to 14 in an amount that enhances delivery of the nucleic acid molecule to a cell relative to a lipid nanoparticle lacking said compound.

78. The lipid nanoparticle of any one of claims 74 to 77, wherein the structural component further comprises one or more structural lipids or salts thereof.

79. The lipid nanoparticle of any one of claims 74 to 78, wherein the one or more structural lipids is a sterol.

80. The lipid nanoparticle of any one of claims 74 to 79, wherein the one or more structural lipids is a phytosterol.

81. The lipid nanoparticle of claim 80, wherein the phytosterol is β-sitosterol, campesterol, sitostanol, stigmasterol, campestanol, fucosterol, or brassicasterol, or any combination thereof.

82. The lipid nanoparticle of any one of claims 74 to 81, wherein the one or more structural lipids is a zoosterol.

83. The lipid nanoparticle of claim 82, wherein the zoosterol is cholesterol.

84. The lipid nanoparticle of any one of claims 78 to 83, wherein the mol % of the one or more structural lipids is between about 1% and 50% of the mol % of the compound of any one of claims 1 to 14 present in the lipid nanoparticle.

85. The lipid nanoparticle of any one of claims 78 to 84, wherein the mol % of the one or more structural lipids is between about 10% and 40% of the mol % of the compound of any one of claims 1 to 14 present in the lipid nanoparticle.

86. The lipid nanoparticle of any one of claims 78 to 85, wherein the mol % of the one or more structural lipids is between about 20% and 30% of the mol % of the compound of any one of claims 1 to 14 present in the lipid nanoparticle.

87. The lipid nanoparticle of any one of claims 78 to 86, wherein the mol % of the one or more structural lipids is about 30% of the mol % of the compound of any one of claims 1 to 14 present in the lipid nanoparticle.

88. The lipid nanoparticle of any one of claims 74 to 87, wherein the lipid nanoparticle comprises one or more non-cationic helper lipids.

89. The lipid nanoparticle of claim 88, wherein the one or more non-cationic helper lipids is a phospholipid, fatty acid, or any combination thereof.

90. The lipid nanoparticle of claim 89, wherein the phospholipid is a phospholipid that comprises a phosphocholine moiety, a phosphoethanolamine moiety, or a phosphor-1-glycerol moiety.

91. The lipid nanoparticle of claim 89 or 90, wherein the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, or 1,2-didocosahexaenoyl-sn-glycero-3-phosphocholine.

92. The lipid nanoparticle of claim 91, wherein the phospholipid is DSPC.

93. The lipid nanoparticle of claim 91 or 92, wherein the phospholipid is 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, or 1,2-dioleoyl-sn-glycero-3-phospho-rac-(1-glycerol) sodium salt (DOPG).

94. The lipid nanoparticle of claim 90, wherein the phospholipid is sphingomyelin.

95. The lipid nanoparticle of claim 89, wherein the fatty acid is a long-chain fatty acid.

96. The lipid nanoparticle of claim 95, wherein the fatty acid is palmitic acid, stearic acid, palmitoleic acid, oleic acid, or any combination thereof.

97. The lipid nanoparticle of claim 96, wherein the fatty acid is oleic acid.

98. The lipid nanoparticle of claim 96, wherein the fatty acid is stearic acid.

99. The lipid nanoparticle of any one of claims 74 to 98, wherein the lipid nanoparticle comprises one or more PEG-lipids.

100. The lipid nanoparticle of claim 99, wherein the one or more PEG-lipids is a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, or mixtures thereof.

101. The lipid nanoparticle of claim 99 or 100, wherein the one or more PEG-lipids is PEG-c-DOMG, PEG-DMG, PEG-DLPE, PEG-DMPE, PEG-DPPC, or PEG-DSPE lipid.

102. The lipid nanoparticle of claim 101, wherein the one or more PEG-lipids is PEG-DMG.

103. The lipid nanoparticle of any one of claims 74 to 102, wherein the lipid nanoparticle comprises about 30 mol % to about 60 mol % one or more ionizable lipids, about 0 mol % to about 30 mol % one or more non-cationic helper lipids, about 18.5 mol % to about 48.5 mol % structural component, and about 0 mol % to about 10 mol % one or more PEG-lipids.

104. The lipid nanoparticle of any one of claims 74 to 103, wherein the lipid nanoparticle comprises about 35 mol % to about 55 mol % one or more ionizable lipids, about 5 mol % to about 25 mol % one or more non-cationic helper lipids, about 30 mol % to about 40 mol % structural component, and about 0 mol % to about 10 mol % one or more PEG-lipids.

105. The lipid nanoparticle of any one of claims 74 to 104, wherein the lipid nanoparticle comprises about 50 mol % one or more ionizable lipids, about 10 mol % one or more non-cationic helper lipids, about 38.5 mol % structural component, and about 1.5 mol % one or more PEG-lipids.

106. The lipid nanoparticle of any one of claims 75 to 105, wherein the nucleic acid molecule is RNA or DNA.

107. The lipid nanoparticle of any one of claims 75 to 106, wherein the nucleic acid is DNA.

108. The lipid nanoparticle of claim 107, wherein the nucleic acid molecule is ssDNA.

109. The lipid nanoparticle of claim 107, wherein the nucleic acid is DNA comprising CRISPR.

110. The lipid nanoparticle of any one of claims 75 to 106, wherein the nucleic acid is RNA.

111. The lipid nanoparticle of claim 110, wherein the nucleic acid molecule is a shortmer, an antagomir, an antisense, a ribozyme, a small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

112. The lipid nanoparticle of claim 110 or 111, wherein the nucleic acid molecule is an mRNA.

113. The lipid nanoparticle of claim 112, wherein the mRNA is a modified mRNA comprising one or more modified nucleobases.

114. The lipid nanoparticle of claim 112 or 113, wherein the mRNA comprises one or more of a stem loop, a chain terminating nucleoside, a polyA sequence, a polyadenylation signal, and a 5′ cap structure.

115. The lipid nanoparticle of any one of claims 74 to 114, wherein the lipid nanoparticle further comprises an additional compound of any one of claims 1 to 14.
